(19) United States
(12) Patent Application Publication
(10) Pub. No.: US 2018/0117084 A1
(43) Pub. Date: May 3, 2018

(54) METHODS FOR ENHANCING AN IMMUNE RESPONSE WITH A CTLA-4 ANTAGONIST

(71) Applicant: BAYLOR COLLEGE OF MEDICINE, Houston, TX (US)

(72) Inventors: Matthew HALPERT, Houston, TX (US); William K. DECKER, Houston, TX (US); Vanaja KONDURI, Houston, TX (US)

(21) Appl. No.: 15/570,799

(22) PCT Filed: Apr. 29, 2016
(86) PCT No.: PCT/US2016/030124

§ 371 Date: Oct. 31, 2017

Related U.S. Application Data
(60) Provisional application No. 62/155,959, filed on May 1, 2015.

(51) Int. Cl. A61K 35/15; A61K 31/713; C07K 16/28; A61K 39/00; C12N 15/113

(52) U.S. Cl. CPC A61K 35/15; C12N 2310/531; C07K 16/2818; A61K 39/0011; C12N 15/1138; C07K 2317/76; C07K 2317/55; C07K 2317/54; C07K 2317/622; C07K 2317/35; C07K 2317/569; C07K 2317/21; C07K 2317/24; A61K 2039/5158; A61K 2039/5154; C12N 2310/14; A61K 31/713

(57) ABSTRACT

Methods are provided for enhancing an immune response comprising providing an immunogenic composition in conjunction with a CTLA-4 antagonist. Dendritic cell populations having reduced CTLA-4 expression are likewise provided.

This application claims the benefit of U.S. Provisional Patent Application No. 62/155,959, filed May 1, 2015, the entirety of which is incorporated herein by reference.

### INCORPORATION OF SEQUENCE LISTING

The sequence listing that is contained in the file named “BACMP0005WO_ST25.txt”, which is 1 KB (as measured in Microsoft Windows®) and was created on Apr. 19, 2016, is filed herewith by electronic submission and is incorporated by reference herein.

### BACKGROUND OF THE INVENTION

### 1. Field of the Invention

The present invention relates generally to the field of molecular biology, immunology an medicine. More particularly, it concerns methods for enhancing an immune response.

### 2. Description of Related Art

Cytotoxic T-lymphocyte antigen 4 (CTLA-4) is a crucial regulator of T-cell immunity in both mice and humans (Krummel and Allison, 1995), the critical importance of which was first demonstrated by the dramatic phenotype of homozygous null mutants which died from massive lymphoproliferative disease and autoimmunity in the postnatal period (Waterhouse et al., 1995; Tivol et al., 1995). Recent reports also demonstrate that heterozygous mutation of human CTLA-4 can result in autosomal dominant immune dysregulation syndrome, underscoring the critical role of CTLA-4 in the maintenance of immune homeostasis (Schubert et al., 2014; Kuehn et al., 2014). In human cancer patients, non-specific antagonism of CTLA-4 has led to immune-mediated cure of advanced cancers, most prominently melanoma (Hodi et al., 2010). CTLA-4 exhibits a complex and controversial biology, with several different hypothesized functions attributed to various alternatively-spliced isoforms. The molecule consists of an extracellular domain that binds B7 (CD80 and CD86) with high affinity, a hydrophobic transmembrane domain, and an intracellular cytoplasmic tail. The current understanding of CTLA-4 function can be broadly divided into cell-intrinsic and cell-extrinsic pathways (Wing et al., 2011). Cell-extrinsic function appears to act by depletion of B7 from the surface of APCs via transendocytosis but may also involve induction of negative signaling in DC (Qureshi et al., 2011; Dejean et al., 2009; Grohmann et al., 2002). Cell-intrinsic function is thought to be less critical to immune homeostasis since CTLA-4 deficient cells in bone marrow chimera with CTLA-4 sufficient cells do not become hyper-activated, yet also likely plays an important role in controlling effector T-cell function by recruitment of SHP-2 and PPA2 negative regulatory phosphatases to the YVKM motif in its cytoplasmic tail. CTLA-4 is also believed to play a role in central tolerance by determining signal strength at the immune synapse during thymic selection (Wing et al., 2011; Qureshi et al., 2011; Kowalczyk et al., 2014; Gardner et al., 2014; Wing et al., 2008). A soluble isoform, often found in the sera of autoimmune disease patients, has also been reported to exist, though the precise function of this isoform has yet to be definitively determined (Esposito et al., 2014; Daroszewski et al, 2009; Purohit et al, 2005; Oaks and Hallett, 2000). Very recent data suggest much of the soluble CTLA-4 detected in acellular sera might actually be full-length CTLA-4 bound to the plasma membrane of secreted microvesicular intermediaries (Esposito et al., 2014).

Though the mechanistic particulars by which CTLA-4 exerts its suppressive activities are an area of substantial debate, its pattern of expression has garnered significantly less controversy. CTLA-4 is thought to exhibit a lymphoid lineage-specific pattern of expression with reports describing expression on regulatory T-cells (Read et al, 2000), activated conventional T-cells (Linsley et al., 1992), induced expression on B-cells (Kuiper et al, 1995), and even a recent report of natural killer (NK) cell expression (Sojanovic et al., 2014). Surface staining does not generally detect CTLA-4 expression on other hematopoietic lineages. Further, transgenic expression of CTLA-4 from a T-cell specific promoter was sufficient to abrogate the lethal autoimmunity observed in CTLA-4-deficient mice, suggesting critical functions of CTLA-4 may be primarily limited to the T-lymphoid lineage (Masteller et al, 2000). However, despite significant mechanist investigation into the functions CTLA-4 it has remained unclear how CLLA-4 function might be modulated to achieve immunological benefit.

### SUMMARY OF THE INVENTION

In a first embodiment there is provided an immunogenic composition comprising at least a first antigen or antigen-primed dendritic cell and a CTLA-4 antagonist. In a further embodiment there is provided a method of providing an immune response in a subject comprising administering an immunogenic composition to the subject in conjunction with a CTLA-4 antagonist.

In some aspects, CTLA-4 antagonist for use according to the embodiments is a small molecule inhibitor or an inhibitor nucleic acid specific to CTLA-4. In certain aspects, the inhibitory nucleic acid is a RNA. In further aspects, the RNA is a small interfering RNA (siRNA) or a short hairpin RNA (shRNA).

In further aspects, a CTLA-4 antagonist is a CTLA-4-binding antibody. In some aspects, the antibody is a monoclonal antibody or a polyclonal antibody. In some aspects, a CTLA-4-binding antibody may be an IgG (e.g., IgG1, IgG2, IgG3 or IgG4), IgM, IgA, genetically modified IgG isotype, or an antigen binding fragment thereof. The antibody may be a Fab′, a F(ab′)2 a F(ab′)3, a monovalent scFv, a bivalent scFv, a bispecific or a single domain antibody. The antibody may be a human, humanized, or de-immunized antibody.

In some aspects, an immunogenic composition of the embodiments comprises an antigen-primed dendritic cell population. In other aspects, the immunogenic composition may comprise a polypeptide antigen. In certain aspects, the immunogenic composition may comprise a nucleic acid encoding an antigen. In particular aspects, the nucleic acid is a DNA expression vector. In alternative aspects of this method, the immunogenic composition may be administered before or essentially simultaneously with the CTLA-4 antagonist or it may be administered after the CTLA-4 antagonist. In specific aspects, the immunogenic composition is administered within about 1 week, 1 day, 8 hours, 4 hours, 2 hours or 1 hour of the CTLA-4 antagonist.

Certain aspects of the embodiments concern immunogenic compositions. In some cases, the immunogenic composition comprises a tumor cell antigen or an infectious disease antigen. In certain aspects, the immunogenic composition comprises at least a first adjuvant. In some aspects, the subject has or is at risk for a disease. In particular aspects, the disease is an infectious disease or a cancer.

In still a further embodiment of the invention, there is provided a dendritic cell population, wherein said population has been has been genetically modified to reduce the expression of CTLA-4. In some aspects, the genetic modification comprises introduction of an exogenous inhibitory nucleic acid specific to CTLA-4. In certain aspects, the inhibitory nucleic acid is a RNA. In further aspects, the RNA is a small interfering RNA (siRNA) or a short hairpin RNA (shRNA). In other aspects, the genetic modification comprises a genomic deletion or insertion in the cell population that reduces CTLA-4. For example, the genetic modification can comprise a genomic edit using a CRISPR/Cas nuclease system. In specific aspects, the cell population comprises a hemizygous deletion within the CTLA-4 gene.

In yet still a further embodiment the invention provides a method of providing an immune response in a subject comprising administering an effective amount of a cell population according to the embodiment and aspects described above. In some specific aspects of this method, the dendritic cells have been primed with at least a first antigen. In certain aspects, the subject has a cancer and the dendritic cells have been primed with at least a first cancer cell antigen. In other aspects, the subject has an infectious disease and the dendritic cells have been primed with at least a first infectious disease antigen.

In certain aspects, the composition comprises an antigen-primed dendritic cell. In other aspects, the composition comprises a first antigen, wherein the antigen is a tumor cell antigen or an infectious disease antigen.

In still a further embodiment of the invention, there is provided a method for culturing antigen specific T-cells comprising culturing a population of T-cells or T-cell precursors in the presence of a dendritic cell population cell population that has been primed with at least a first antigen, wherein (i) said culturing is in the presence of a CTLA-4 antagonist or (ii) said dendritic cell population has been has been genetically modified to reduce the expression of CTLA-4. In some aspects, the method is further defined as a method for ex vivo expansion of antigen specific T-cells. In certain aspects, the dendritic cell population comprises primary dendritic cells. In further aspects, said culturing is in the presence of a CTLA-4 antagonist. In specific aspects, the CTLA-4 antagonist is an inhibitor nucleic acid specific to CTLA-4. In certain aspects, the inhibitory nucleic acid is a RNA. In further aspects, the RNA is a small interfering RNA (siRNA) or a short hairpin RNA (shRNA). In other aspects, the CTLA-4 antagonist is a CTLA-4-binding antibody.

In still further aspects of this method, said dendritic cell population has been has been genetically modified to reduce the expression of CTLA-4. In some aspects, the genetic modification comprises introduction of an exogenous inhibitory nucleic acid specific to CTLA-4. In certain aspects, the inhibitory nucleic acid is a RNA. In particular aspects, the RNA is a small interfering RNA (siRNA) or a short hairpin RNA (shRNA). In some specific aspects, the genetic modification comprises a genomic deletion or insertion in the cell population that reduces CTLA-4. In other aspects, the cell population comprises a hemizygous or homozygous deletion within the CTLA-4 gene. For example, in some aspects, one or both copies of the CTLA-4 gene of a dendritic cell can be completely or partially deleted, such that expression the CTLA-4 polypeptide is inhibited.

Aspects of the embodiments concern compositions and methods for treating disease in a subject. For example, the disease can be an infectious disease or a cancer. In some aspects, the cancer may be a breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer or skin cancer. A subject for treatment according to the embodiments is, in some aspects, a mammalian subject. For example, the subject may be a primate, such a human. In further aspects, the subject is a non-human mammal, such as a dog, cat, horse, cow, goat, pig or zoo animal.

Certain aspects of the embodiments concern administration of cell compositions or immunogenic compositions to a subject. In one aspect, the composition may be administered systemically. In additional aspects, the composition may be administered intravenously, intradermally, intratumorally, intramuscularly, intraperitoneally, subcutaneously, or locally. The method may further comprise administering at a second therapy to the subject. For example, in some aspects, the second therapy is an anticancer therapy. Examples of the second anticancer therapy include, but are not limited to, surgical therapy, chemotherapy, radiation therapy, cryotherapy, hormonal therapy, immunotherapy, or cytokine therapy.

In further aspects, a method of the embodiments may further comprise administering a composition of the present invention more than one time to the subject, such as, for example, 1, 2, 3, 4, 5, 6, 7, 8, 9, 10, 15, 20 or more times.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B—Dendritic cells secrete CTLA-4. (A) Human DC were differentiated (GM-CSF, IL-4) from the adherent fraction of a buffy coat with or without prior CD14-selection and CD11c-enrichment and subsequently matured with IL-13, IL-6, TNFα, and PGE₂ for 48 hours. DC were also treated with either non-targeting (NT) siRNA or CTLA-4 siRNA at time of maturation, and DC-cultured supernatants were collected and assayed for CTLA-4 compared to variously stimulated non-adherent PBMC derived from the same buffy coat. (B) DC-cultured supernatants were rotated overnight at 4° C. with protein G-plus beads coated with either the BNI3 clone or A3.6B10.G1 clone of αCTLA-4, or an isotype control antibody. IL-12p35 was used to validate antibody coIP specificity.

FIGS. 2A-E—Dendritic cells possess intracellular CTLA-4. Following CD14-selection, DC-differentiation, and CD1c enrichment, DC were analyzed for intracellular CTLA-4. CD11c⁺ DC were shown to possess intracellular CTLA-4 by (A) flow cytometry, (B) immunofluorescent confocal microscopy, and (C) RT-PCR. All methods revealed an increase in CTLA-4 quantity corresponding to DC maturation, and CTLA-4 siRNA successfully reduced CTLA-4 mRNA levels. (D) DC displayed a more global distribution of CTLA-4 than polarized, surface-bound CTLA-4 associated with T-cells. (E) Tolerogenic DC differentiated with M-CSF and TGF-β possessed higher levels of intracellular CTLA-4 than conventional GM-CSF/IL-4 differentiated DC.

FIGS. 3A-E—Dendritic cells secrete full-length CTLA-4 packaged within microvesicular structures. (A) DC-cultured supernatants were pre-cleared with naked protein G-plus beads and subsequent coIP with anti-CD63-coated beads. Depleted supernatants were then analyzed by western blot for full-length CTLA-4 (flCTLA-4) content. Alternatively, supernatants were treated with various concentrations of NP-40 for one hour prior to coIP and then analyzed by western blot for flCTLA-4 remaining in the supernatants. (B, C) Immature and mature DC were analyzed by confocal microscopy to identify Golgi apparatus, Rab5, and CTLA-4 localization. (D) DC-culture supernatants derived from three independent buffy coat products were treated with the Invitrogen Total Exosome Isolation Reagent. Purified exosomes (30-120 nm) were compared by western blot to remaining supernatant components for CD63, Rab5, IL-12, and CTLA-4. (E) Exosomes purified from DC-cultured supernatants were incubated with anti-CTLA-4 coated beads. The CTLA-4⁺ pull-down fraction was then compared to the residual fraction by western blot for CTLA-4, CD63, Rab5, and Rab11. *p<0.05.

FIGS. 4A-E—DC-derived exosomes are internalized by DC in an autocrine/paracrine fashion mediated by exosome surface CTLA-4. (A) Staining pattern of CFSE-labeled DC indicating some colocalization with CTLA-4 structures. (B) Cultured supernatants from CFSE loaded DC were subsequently depleted of all cells and incubated with unlabeled DC for various time points. Recipient, unlabeled DC could be visualized binding and (C) internalizing CFSE⁺ microvesicles. (D) Cultured CFSE-loaded DC supernatants were incubated with protein G-plus beads coated with various concentrations of αCTLA-4, and treated supernatants were subsequently incubated with unlabeled DC for 6 hrs @ 37° C. before flow cytometric analysis of CFSE⁺ microvesicle uptake. (E) Recipient DC were also analyzed for their ability to still bind αCD86 and αCD80 antibodies after 6 hour and 12 hour incubations with CFSE⁺ CTLA-4-microvesicles. A significant log-fold decrease in B7 expression was apparent among DC that internalized CFSE⁺ microvesicles. This decrease was specific to B7 and not observed among other markers such as CD11c. Error bars at 6 and 12 hours=+/−SD of four independent experiments. *=p<0.05.

FIGS. 5A-B—siRNA knockdown of CTLA-4 in CFSE-loaded DC diminish uptake of CFSE-loaded exosomes by unlabeled recipient DC. (A, B) DC were loaded with 5 μM CFSE, treated with CTLA-4 or non-targeting (NT) siRNA for 72 hours, and matured. Culture supernatants were then collected and incubated with unlabeled DC for various lengths of time before flow cytometric analysis for levels of CFSE uptake and residual ability of CD80 (B7-1) to still be stained by specific antibodies.

FIGS. 6A-D—Knockdown of DC CTLA-4 Enhances the T_{H}1 response and anti-tumor immunity. (A) Human DC were treated with CTLA-4 or non-targeting (NT) siRNA for 72 hours, matured, and cocultured at a ratio of 1:10 with syngeneic T-cells with restimulation on days 9 and 24. T-cells were sampled throughout the process by incubation in brefeldin A for five hours and analysis by flow cytometry to determine CD4:CD8 ratio, CD8 activation (CD25 and intracellular IFN-γ), and quantitation of CD4+CD25+Foxp3+ tregs. Data shown are representative of five independent experiments with five biologically distinct products. (B) Relative CTLA-4 concentrations of various siRNA-treated mouse DC culture supernatants was characterized by western blot after which 1×10⁶ total splenocytes were cultured in these supernatants with supplemental IL-2 added on days 5, 7, and 9. The data indicated that that proliferation of CD8+CD25+ cells was dependent upon low levels of CTLA-4 supernatant content as well as proportional to the concentration of CTLA-4 in the supernatant. (C/D) Mouse BMDC were differentiated from mouse bone marrow cultured with GM-CSF and IL-4 for 6 days, treated with CTLA-4 or non-targeting (NT) siRNA for 72 hours, loaded with B16 mRNA, matured, and injected into the ipsilateral footpad of recipient C57BL/6 mice in which palpable B16 tumors had been pre-established 3 days prior. Mice were boosted on day 14, and tumors were measured routinely for >3 weeks. Cohorts consisted of five mice each. *p<0.05. (E) DC were polarized during in vitro maturation toward either TH1 or TH2, and culture supernatants were analyzed for the presence of DC-secreted CTLA-4 by Western blot after 24 hours. TH1=polarized with 1 ng/ml IL-12. TH2=polarized with 10 ng/ml (1×) or 100 ng/ml (10×) SEB. IM=immature DC.

FIG. 7—Common animal sera do not exhibit detectable presence of full-length CTLA-4. Media made with various common sera (mouse, human, and bovine) were analyzed for CTLA-4 prior introduction of fresh DC to determine potential for pre-existing contamination. PBMC lysate was used as a CTLA-4 western blot control.

FIGS. 8A-B—Though T-cells did not secrete detectable CTLA-4, they were appropriately activated as determined by CFSE proliferation assay, upregulation of CD25, and IFN-γ Secretion. (A, B) To confirm that the lack of CTLA-4 secreted from T cells was not due to insufficient stimulation, T cell activation was measured by CFSE proliferation and CD25 upregulation (flow cytometry), and IFN-γ secretion (western blot).

FIG. 9—DC purity after CD14-selection, differentiation, and CD11c-enrichment. The CD14⁺ monocytic fraction of the buffy coat was magnetically selected prior to DC differentiation and subsequent CD11c-enrichment before analysis for CD3⁺ cell contamination, and before subsequent use in experimentation.

FIG. 10—Functional validation of CTLA-4 siRNA specificity. CTLA-4 or non-targeting (NT) siRNA was electroporated into T-cells 48 hours prior to analysis. CTLA-4 knockdown was subsequently validated by western blot and upregulation of T-cell CD25 expression.

FIG. 11—Intracellular DC CTLA-4 is upregulated with increased DC maturation. Intracellular CTLA-4 levels were well-correlated with relative maturity of the DC as measured by CD80 and CD83 expression.

FIG. 12—Circulating human CD1c⁺ cells physiological express intracellular CTLA-4 in a pattern distinct from that of CD3⁺ cells. Blood was collected from healthy volunteers and subsequently stained for CD11c, CD3, and intracellular CTLA-4. Gating specifically on CD3⁺ and CD11c⁺ cells indicates that, while activation with SEB upregulates CTLA-4 expression on the CD3⁺ subset (top panel), both subsets possess intracellular CTLA-4 (bottom panel). Basal levels of intracellular CTLA-4 were higher in CD11c⁺ cells than in unactivated CD3⁺ cells.

FIG. 13—Validation of αCTLA-4 antibody specificity. T-cells were stained with either αCTLA-4 or isotype control antibody and analyzed by confocal microscopy to confirm αCTLA-4 antibody specificity.

FIGS. 14A-C—CTLA-4 siRNA targets DC CTLA-4 though downregulation of protein levels is delayed. CTLA-4 siRNA targets DC CTLA-4 and ultimately leads to protein reduction as assayed by (A, B) western blot and (C) confocal microscopy. Unlike T-cells which show virtual complete loss of protein 48 hours after CTLA-4 siRNA treatment, DC CTLA-4 exhibits greater stability with little reduction in protein levels until 72 hours post-treatment.

FIG. 15—Rab11 and CTLA-4 do not colocalize in DC. DC were analyzed by immunofluorescent confocal microscopy to determine localization of Rab11 and CTLA-4.

FIG. 16—Quantitation of FIG. 4D—Incubation of DC cell culture supernatants with αCTLA-4 coated beads blocks subsequent uptake of CFSE-labeled exosomes by unlabeled DC. Incubation of DC cell culture supernatants with αCTLA-4 coated beads blocked the subsequent uptake of CFSE-labeled exosomes by unlabeled DC in a titratable fashion. At an antibody concentration of 5 μg/ml, 26% fewer recipient DC internalized CFSE⁺ exosomes (p=0.02*) whereas at an antibody concentration of 50 μg/ml, 35% fewer recipient DC internalized CFSE⁺ exosomes (p=0.007**). Staining of CD11c internal control was not statistically different between groups. Error bars=+/−SD of three independent experiments.

FIGS. 17A-B—DC-exosome CTLA-4 physiologically binds B7. (A) CFSE-loaded DC culture supernatants were serially diluted with fresh media and incubated with unlabeled DC for 20 minutes @ 4° C. along with αCD80 and αCD86 flow-qualified antibodies and 0.1% sodium azide. (B) Similar to FIGS. 4D and 4E, cultured CFSE-loaded DC supernatants were incubated for 1 hour with protein G-plus beads coated with various concentrations of αCTLA-4. The beads were pelleted and cleared supernatants were incubated with unlabeled DC for 20 minutes @ 4° C. along with αCD80 and αCD86 flow-qualified antibodies and 0.1% sodium azide. CD11c was used as a non-B7 control.

FIG. 18—Quantitation of FIG. 17A—DC-exosome CTLA-4 physiologically binds B7. In three experiments, incubation of unlabeled recipient DC with cell culture supernatants derived from CFSE-labeled DC for 20 minutes @ 4° C. along with αCD80 and αCD86 flow-qualified antibodies and 0.1% sodium azide resulted in no change in B7 MFI using 1% or 10% supernatant (as well as no detectable uptake of CFSE⁺ vesicles); however, in 100% supernatant, CD80 MFI and CD86 MFI were both significantly reduced as CFSE uptake quintupled. There was no statistical difference in CD11c MFI among any of the dilutions. *=p<0.05. Error bars=+/−SD.

FIG. 19—Quantitation of FIG. 17B—DC-exosome CTLA-4 physiologically binds B7. In three experiments, pre-clearance of CFSE-labeled DC cell culture supernatants with αCTLA-4 beads for one hour prior to incubation of unlabeled DC for 20 minutes @ 4° C. along with αCD80 and αCD86 flow-qualified antibodies and 0.1% sodium azide resulted in a titratable and statistically significant increase in B7 MFI with no concomitant increase in CD11c MFI while percentage of CFSE+ cells dropped 45%. *=p<0.05. Error bars=+/−SD.

FIG. 20—siRNA Knockdown of DC CTLA-4 Enhances Production of CD8⁺ T-cells In Vitro. Quantitation of data from FIG. 6A. In eight independent experiments performed with eight different biological products (buffy coats), percent generation of CD8⁺ T-cells was doubled when autologous PBMC were expanded with CTLA-4 siRNA-treated DC in comparison to non-targeting (NT) siRNA-treated DC. Error bar=+/−SD. p=0.005 by Student's paired t-test.

FIG. 21—Analysis of cells derived from CTLA-4−/−CD28−/− double knockout mice confirms expression of CTLA-4 in murine DC. Splenocytes, BMDC, and BMDC culture supernatants were generated from wild type and CTLA-4−/−CD28−/− double knockout mice and then analyzed for CTLA-4 content by Western blot analysis. Anti-actin was used as a loading control.

### DESCRIPTION OF ILLUSTRATIVE EMBODIMENTS

### I. The Present Embodiments

The effect of CTLA-4 on antigen presentation was previously not characterized and, accordingly, it was unknown how modulation of CTLA-4 activity might be modulated to control immune response. Studies presented herein demonstrate for the first time that mature myeloid dendritic cells upregulate the expression of intracellular CTLA-4, which is subsequently secreted into the extracellular space (by means of a vesicular intermediary). DC-derived, extracellular CTLA-4 competitively inhibits antibody binding of B7, and its presence negatively regulates downstream T-cell responses in vitro and antitumor immunity in vivo. Thus, the unexpected presence of functional CTLA-4 in this critical hematopoietic lineage indicates an additional level of DC control over the adaptive immune response that could be modulated by CTLA-4 antagonist drugs. In particular, the studies presented here indicate that a CTLA-4 antagonist could be used to enhance T-cell immune response.

Accordingly, embodiments of the present invention provide immunogenic compositions (e.g., vaccine compositions) that include a CTLA-4 antagonist. The addition of such an antagonist enhances the ability of the composition to provide a robust immune response, in particular a robust T-cell mediated immune response. Likewise, provided are methods for providing an enhanced immune response in a subject by administering an immunogenic composition (e.g., a composition comprising an antigen) in conjunction with administration of a CTLA-4 antagonist.

Moreover, in view of the regulatory role of CTLA-4 in dendritic cell antigen presentation, modification CTLA-4 activity can be used to enhance dendritic cell function. Thus, in some aspects, dendritic cells are provided that comprise down-regulated CTLA-4 expression. Importantly, once primed against an antigen, such modified dendritic cells are able to provide a more robust T-cell response. Accordingly, modified dendritic cells provided herein can be primed with antigen and directly administered to a subject to provide an immune response in the subject. Likewise, modified dendritic cells can be used ex vivo to stimulate and expand populations of targeted T-cells, which may in turn be used as a therapeutic.

### II. CTLA-4 Antagonists

Certain aspects of the embodiments concern CTLA-4 antagonists. In some aspects the CTLA-4 antagonist is a small molecule antagonist. In further aspects, the CTLA-4 antagonist can be an antibody that binds to CTLA-4 and prevents its activity. In yet further aspects, a CTLA-4 antagonist can be an inhibitory nucleic acid that reduces CTLA-4 expression.

A. CTLA-4-Binding Antibodies

In certain embodiments, an antibody or a fragment thereof that binds to at least a portion of CTLA-4 protein and inhibits CTLA-4 signaling is contemplated. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand.

Preferably, the anti-CTLA-4 antibody is a monoclonal antibody or a humanized antibody. Thus, by known means and as described herein, polyclonal or monoclonal antibodies, antibody fragments, and binding domains and CDRs (including engineered forms of any of the foregoing) may be created that are specific to CTLA-4 protein, one or more of its respective epitopes, or conjugates of any of the foregoing, whether such antigens or epitopes are isolated from natural sources or are synthetic derivatives or variants of the natural compounds.

Examples of antibody fragments suitable for the present embodiments include, without limitation: (i) the Fab fragment, consisting of VL, VH, CL, and CH1 domains; (ii) the “Fd” fragment consisting of the VH and CH1 domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VH domain and a VL domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulphide bridges linking the VH and VL domains. Minibodies comprising a scFv joined to a CH3 domain may also be made (Hu et al., 1996).

Antibody-like binding peptidomimetics are also contemplated in embodiments. Liu et al. (2003) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as a CTLA-4 polypeptide sequence, in order to produce antibodies specific for CTLA-4 protein. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody is a single species of antibody wherein every antibody molecule recognizes the same epitope because all antibody producing cells are derived from a single B-lymphocyte cell line. The methods for generating monoclonal antibodies (MAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a CTLA-4 antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced.

Plasma B cells (CD45+CD5-CD19+) may be isolated from freshly prepared rabbit peripheral blood mononuclear cells of immunized rabbits and further selected for CTLA-4 binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. CTLA-4 specific binding Fab may be selected out through multiple rounds enrichment panning and sequenced. Selected CTLA-4 binding hits may be expressed as full length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in: Marks (1992); Stemmer (1994); Gram et al. (1992), Barbas et al. (1994); and Schier et al. (1996).

It is fully expected that antibodies to CTLA-4 will have the ability to neutralize or counteract the effects of CTLA-4 regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described above, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 10⁰% may be an antibody that binds CTLA-4.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against CTLA-4, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6?-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

B. CTLA-4 Inhibitory Nucleic Acids

In certain aspects methods involve the use of an inhibitor of CTLA-4 such as an inhibitory nucleic acid that targeted CTLA-4. Examples of inhibitory nucleic acids include, without limitation, antisense nucleic acids, small interfering RNAs (siRNAs), double-stranded RNAs (dsRNAs), microRNAs (miRNA) and short hairpin RNAs (shRNA) that are complimentary to all or part of CTLA-4 mRNA. An inhibitory nucleic acid can, for example, inhibit the transcription of a gene in a cell, mediate degradation of an mRNA in a cell and/or inhibit the translation of a polypeptide from a mRNA. Typically an inhibitory nucleic acid may be from 16 to 1000 or more nucleotides long, and in certain embodiments from 18 to 100 nucleotides long. In certain embodiments, the inhibitory nucleic acid may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some aspects an inhibitory nucleic acid may comprise one or more modified nucleotide or nucleic acid analog. Typically, an inhibitory nucleic acid will inhibit the expression of a single gene within a cell; however, in certain embodiments, the inhibitory nucleic acid will inhibit the expression of more than one gene within a cell.

In some aspects an inhibitory nucleic acid can form a double-stranded structure. For example, the double-stranded structure may result from two separate nucleic acid molecules that are partially or completely complementary. In certain embodiments, the inhibitory nucleic acid may comprise only a single nucleic acid or nucleic acid analog and form a double-stranded structure by complementing with itself (e.g., forming a hairpin loop). The double-stranded structure of the inhibitory nucleic acid may comprise 16 to 500 or more contiguous nucleobases. For example, the inhibitory nucleic acid may comprise 17 to 35 contiguous nucleobases, more preferably 18 to 30 contiguous nucleobases, more preferably 19 to 25 nucleobases, more preferably 20 to 23 contiguous nucleobases, or 20 to 22 contiguous nucleobases, or 21 contiguous nucleobases that are complementary to a CTLA-4 mRNA. Methods for using such siRNA or double-stranded RNA molecules have been described in U.S. Pat. Nos. 6,506,559 and 6,573,099, as well as in U.S. Applications 2003/0051263, 2003/0055020, 2004/0265839, 2002/0168707, 2003/0159161, 2004/0064842, each of which are herein incorporated by reference in their entirety.

### III. Dendritic Cell Populations of the Embodiments

Methods for isolating culturing and priming dendritic cells are well known in the art. For example, U.S. Pat. No. 8,728,806, which is incorporated herein by reference in its entirety, provides detailed methods for providing antigen primed dendritic cells that may be used in the compositions and methods of the embodiments.

A. Genetically Modified Dendritic Cells

Certain aspects of the embodiments concern dendritic cells that have been genetically modified to reduce the expression of CTLA-4. In some aspects, the genetic modification comprises introduction of an exogenous inhibitory nucleic acid specific to CTLA-4. In certain aspects, the inhibitory nucleic acid is a RNA, such as a RNA that is expressed from a DNA vector in the dendritic cells. In further aspects, the inhibitory nucleic acid may be a siRNAs, dsRNA, miRNA or shRNA that is introduced in the dendritic cells. A detailed disclosure of such RNAs is provided above.

In further aspects, the genetic modification comprises a genomic deletion or insertion in the cell population that reduces CTLA-4. In other aspects, the dendritic cells comprises a hemizygous or homozygous deletion within the CTLA-4 gene. For example, in some aspects, one or both copies of the CTLA-4 gene of a dendritic cell can be completely or partially deleted, such that expression the CTLA-4 polypeptideis inhibited. In some aspects, modification the cells so that they do not express one or more CTLA-4 gene may comprise introducing into the cells an artificial nuclease that specifically targets the CTLA-4 locus. In various aspects, the artificial nuclease may be a zinc finger nuclease, TALEN, or CRISPR/Cas9. In various aspects, introducing into the cells an artificial nuclease may comprise introducing mRNA encoding the artificial nuclease into the cells.

Thus, in some embodiments, a genomic modification (e.g., a deletion of edit of the genome) is carried out using one or more DNA-binding nucleic acids, such as disruption via an RNA-guided endonuclease (RGEN). For example, the disruption can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derived from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known, for example, the amino acid sequence of S. pyogenes Cas9 protein may be found in the SwissProt database under accession number Q99ZW2, incorporated herein by reference.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

The reagents used in the examples described below were as follows: Western blot and coIP antibodies—αHuman CTLA-4 clone A3.6B10.G1 (cat #: 525401, Biolegend, San Diego, Calif.); αHuman CTLA-4 clone BNI3 (cat #: 555851, BD Pharmingen, San Diego, Calif.); αHuman CTLA-4 (cat #: ab107198, Biolegend, Cambridge, Mass.); Human/Mouse αIL-12p35 (cat #: MAB1570, R&D, Minneapolis, Minn.); Human αIFNγ (cat #: ab9657, Abcam, Cambridge, Mass.); Protein G Plus Agarose Suspension (cat #: 1P04, Calbiochem, Billerica, Mass.); Human TruStain FcX™ FC block (cat #: 422301, Biolegend, San Diego, Calif.); Ponceau S Solution (cat #: 6226-79-5, Sigma, St. Louis, Mo.); Restore™ Western Blot Stripping Buffer (cat #: 21059, Pierce, Rockford, Ill.); Secondary Antibodies (αGoat-HRP, αRabbit-HRP, αMouse-HRP) and β-actin (Santa Cruz, Dallas, Tex.). Functional antibodies—αHuman CD3 clone UCHT1 (cat #: 555329, BD Pharmingen, San Diego, Calif.); αHuman CD28 (cat #: 555725, BD Pharmingen, San Diego, Calif.). Flow antibodies—αHuman CDJ IC, CD80, CD83, CD86, CD3, CD4, CD8, CD25, IFNγ, and CTLA-4 flow antibodies (Biolegend, San Diego, Calif.). HIV Tetramer (HIV-pol468; ILKEPVHGV) from the Baylor College of Medicine Tetramer Core (Houston, Tex.). Confocal microscopy antibodies and reagents—αCTLA-4-biotin clone BNI3 (cat #: 555852, BD Pharmingen, San Jose, Calif.); Streptavidin-APC (cat #: 554067, BD Pharmingen, San Jose, Calif.); Rab5 (cat #: 108011, mouse-monoclonal synaptic systems, Germany); Rab11 (cat #: 610656 BD Biosciences); Giantin (Courtesy: Dr. Rick Sifers, BCM, Houston, Tex.); Alexa-fluor Ms546 (Courtesy: Dr. Anna Sokac, BCM, Houston, Tex.); Alexa-fluor Rb546 (Courtesy: Dr. Anna Sokac, BCM, Houston, Tex.); CD3-FITC (cat #: 555332, BD Pharmingen, San Jose, Calif.); CD11c clone 3.9-Alexa-fluor 488: (cat #: 301618, Biolegend, San Diego, Calif.); DAPI: SlowFade® Gold Antifade Mountant (cat #: S36938, Molecular Probes, Grand Island, N.Y.). Dendritic cell selection and enrichment—Human CD14 Positive Selection Kit (cat #: 18018, EasySep, Stemcell Technologies, Vancouver, Canada); Human Myeloid DC Enrichment Kit (cat #: 19021, EasySep, Stemcell Technologies, Vancouver, Canada).

Four-to-six-week-old C57BL/6 mice were obtained from Baylor College of Medicine (Houston, Tex.). All mice were maintained in accordance with the specific IACUC requirements of Baylor College of Medicine.

Dendritic Cell Preparation, Enrichment, and Maturation.

Normal donor peripheral blood buffy coats were obtained from the Gulf Coast Regional Blood Bank. Products were diluted 1:3 in PBS (Lonza, Allendale, N.J.) and centrifuged at 450×g on a Ficoll gradient (Lympholyte, Cedarlane Labs, Burligton, N.C.) to isolate viable white cells. CD14⁺ cells were magnetically separated from total PBMC using Clinimacs CD14 beads (Miltenyi-Biotec, San Diego, Calif.) according to the manufacturer's instructions. CD14⁺ cells were cultured for 6 days in AIM-V medium (Invitrogen, Carlsbad, Calif.) supplemented with 10% Human AB Serum (Atlanta Biologicals, Lawrenceville, Ga.), 50 μg/ml streptomycin sulfate (Invitrogen), 10 gig/ml gentamicin sulfate, 2 mM 1-glutamine (Invitrogen), 50 ng/ml GM-CSF (Amgen, Thousand Oaks, Calif.), and 10 ng/ml IL-4 (R&D Systems, Minneapolis, Minn.). The culture medium was removed and replenished with an equal volume of fresh medium on day 3. Cells were cultured in a humidified chamber at 37° C. and 5% atmospheric CO₂. On day 6 of differentiation, immature DC were harvested and further enriched using the EasySep Human Myeloid DC Enrichment Kit (StemCell Technologies, Vancouver, BC) according to the manufacturer's instructions. If matured, DC were cultured for an additional 48 hours in AIM-V supplemented as previously described but with the addition of ITIP [10 ng/ml IL-1β (R&D Systems), 10 ng/ml TNF-α (R&D Systems), 15 ng/ml IL-6 (R&D Systems), and 1 μg/ml PGE₂ (Sigma)].

Tolerogenic Dendritic Cells.

Buffy coat DC were prepared from adherent monocytes and incubated as previously described, but were differentiated in the presence of 100 ng/ml macrophage colony-stimulating factor (M-CSF) and 10 ng/ml TGF-β (eBioscience, SD, Calif.). Differences from conventional DC preparations were verified by flow cytometry of CD11c, CD80, CD83, and CD86.

T-Cell Stimulation and Analysis.

PBMC were isolated from the non-adherent fraction of a Buffy Coat, resuspended in RPMI-10/o FBS, 1% anti-anti, loaded with 1 uM CFSE, and plated at 1×10⁶/well in a 96-well immunoabsorbent flat-bottom plate previously coated (24 hours, 4° C.) with 1 ug/ml immobilized αCD3 (clone UCHT1). The plate was incubated at 37° C., 5% CO₂ for 3 days, cells were washed with PBS, re-plated in a 96-well round bottom plate at 10⁵ cells/well and treated with αCD28 (various concentrations) for 3 days at 37° C., 5% CO₂. Alternatively, PBMC were treated for 4 days with various concentrations of SEB. Cells were then analyzed by flow cytometry for CFSE levels, and cultured supernatants were analyzed by western blot for IFN-γ.

siRNA Transfection.

CTLA-4 (mouse and human) siGenome SMART Pools and non-targeting siRNA pools were purchased from Thermo Scientific (Wilmington Del.). In brief, siRNA was reconstituted in 50 μl of siRNA buffer, and 1 ul/reaction was pre-diluted in Viaspan (Barr Laboratories subsidiary of Teva Pharmaceuticals, Pomona, N.Y.) before 1:1 addition to cells resuspended in Viaspan (20-40×10⁶/ml). Cells were incubated on ice for 10 min prior to electroporation (DC—250 V, 125 μF, Ω=∞, 4 mm cuvette; T-cell—140V, 1000 μF, Ω=∞, 4 mm cuvette) using a Gene Pulser Xcell Electroporator (Bio-rad, Hercules, Calif.).

Western Blotting and Analysis.

All gel electrophoresis was performed under denaturing, reducing conditions on a 12% polyacrylamide gel with subsequent transfer to an 0.45 μm nitrocellulose membrane for antibody probing. All blocking and antibody staining steps were carried out in 5% milk, and primary antibodies were applied overnight at 4° C. Western blot chemiluminescent signal was detected using a ChemiDoc XRS digital imaging system supported by Image Lab software Version 2.0.1 (Bio-Rad Laboratories, Hercules, Calif.). All Western blots were quantitated by densitometry of Ponceau S (Sigma-Aldrich) stained membranes. Contamination of supernatants with residual cell lysate or debris from cell death was controlled for by immunostaining with anti-β-actin (Santa Cruz) and additional densitometry. Densitometry was performed using ImageJ software (NIH; Bethesda, Md.). All western blots are representative of at least 3 independent experiments.

Co-Immunoprecipitation.

Samples were prepared based on individual experimental approach. Either cultured media supernatant was separated from cells via centrifugation (400×g, room temperature, 5 minutes) or cells were lysed with various concentrations of NP-40 Lysis buffer (1 hour, 4° C.) followed by centrifugation of debris (20 minutes, 40 C, 20,000×g). Samples were then pre-cleared with naked Protein G plus beads (1 hour, room temperature with rotation), followed by centrifugation of the beads (10 minutes, room temperature, 100×g). The remaining supernatant was then incubated with αCTLA-4-coated beads (overnight, 4° C. with rotation), followed by centrifugation of the beads (10 minutes, room temperature, 100×g). Beads were then carefully washed 5× with either PBS or detergent, and the remaining contents of the Protein G pulldown were boiled in SDS- and β-mercaptoethanol-containing gel electrophoresis loading dye and subsequently analyzed by western blot.

Flow Cytometry and Analysis.

All flow cytometric analysis was performed using an LSR II flow cytometer (BD Biosciences) and analyzed with FlowJo version 10.0.00003 for the MacIntosh (Tree Star Inc, Ashland, Oreg.). All flow analyses shown are representative of at least 3 independent experiments.

Immunofluorescence and Confocal Microscopy.

Dendritic cells were cultured and matured in a 6-well plate and subsequently collected onto 12 mm round poly-L-lysine coated cover slips (Corning Inc) in a 24-well plate by centrifugation (400×g, room temperature, 5 minutes). The media was aspirated and cells were gently washed 2× with ice-cold PBS. The cells were fixed in 4% formaldehyde in PEM (80 mM Potassium PIPES pH 6.8, 5 mM EGTA pH 7.0 and 2 mM MgCl₂—all from Sigma) buffer for 30 min on ice. After fixation, the cells were washed 3× with PEM buffer (5 min/wash). To quench autofluorescence and enhance antigenicity, the coverslips were incubated 2× for 5 minutes in 1 mg/ml freshly made sodium borohydride (Sigma) in PEM buffer. Quenching was followed by washing the cells 2× with PEM buffer. The cells were then permeabilized by incubating the coverslips in PEM+0.5% Triton-X-100 (ThermoFisher Scientific, Waltham, Mass.) for 30 minutes. The cells were washed 3× with PEM buffer (5 minutes/wash). Blocking was performed with TBS-T/1% BSA (Sigma) (1 hour, room temperature). The blocking buffer was removed, appropriate primary antibody was added and the cells were incubated in the primary antibody overnight at 4° C. Primary antibody was removed and the cells were washed 5× in the blocking buffer followed by incubation in the appropriate secondary antibody (1 hour, room temperature). The secondary antibody was removed followed by five TBS-T and two PEM washes (5 minutes each). The cells were then fixed in 4% formaldehyde in PEM for 20 min followed by 3 PEM washes (5 minutes). To quench autofluorescence, the coverslips were incubated 2× with 1 mg/ml freshly made sodium borohydride in PEM buffer followed by 2 washes with PEM and 2 washes with TBS-T. The cells were then counterstained with DAPI (Molecular Probes division of LifeTechnologies, Grand Island, N.Y.) for 2 minutes. DAPI was removed and TBS-T was added to the cells. The coverslips were mounted on the slides using Prolong® Gold antifade reagent (Molecular Probes). Image acquisition was performed on a Zeiss LSM 710 confocal microscope with a 60×/0.95 numerical aperture oil-immersion objective (Carl Zeiss, Inc, Peabody, Mass.). Images were collected at a zoom factor of two with a resolution of 104 nm per pixel. Antibodies used were: CTLA-4-biotin (0.25 μg/ml) with streptavidin-APC (1:500), Rab5 (1:1000) with Alexa-fluor Ms546 (1:500), Giantin (1:1000) with Alexa-fluor Rb546 (1:500), CD3-FITC (1:10), CD11c-Alexa-fluor 488 (2.5 ug/mL) and DAPI (1:2500). All images shown are representative of at least 3 independent experiments.

CTLA-4 RT-PCR.

Loaded, matured DC were resuspended in 1 ml Trizol (Life Technologies) at <1×10⁷ cells per sample and total RNA was extracted according to manufacture's instructions. RNA was treated with 1 μg/μl DNase I (Invitrogen). cDNA was synthesized from the DNase-treated RNA sample using the SuperScript™ III First-Strand Synthesis kit (Life Technologies) and amplified by PCR for 35 cycles at an annealing temperature of 55° C. with CTLA-4 Fwd primer: ATGGCTTGCCTTGGATTTCAGCGGC (SEQ ID NO: 1) and CTLA-4 Rev primer: TCAATTGATGGGAATAAAATAAGGCTG (SEQ ID NO: 2). Primers were designed to amplify transcripts corresponding to both soluble and membrane-bound CTLA-4 isoforms. GAPDH was amplified as a control.

In Vitro Co-Culture.

Dendritic Cells were treated with either CTLA-4 or non-targeting siRNA for a total of 72 hours and matured for a total of 48 hours prior co-culture setup with autologous T cells at a ratio of 1:10 in RPMI-1640/10% FBS/1^{%} anti-anti and incubated at 37° C. in 5% atmospheric CO₂. T-cells were restimulated with appropriate DC on day 9, and recombinant human IL-2 at 200 IU/ml (Chiron subsidiary of Novartis, Emeryville, Calif.) was added to the culture on days 5, 7, 10, 12, 14, 16, 18, 20, 22 with corresponding expansion allowance. T-cells were collected at various time points for proliferative counts and flow cytometric analysis.

In Vivo B16 Tumor/Vaccination.

Mouse bone marrow derived DC (BMDC) were prepared as follows: Bone marrow was isolated from C57BL/6 mouse femurs and tibias, red blood cells were lysed with ACK lysing buffer (LifeTechnologies) for 5 minutes at room temperature, and the remaining cells were resuspended in 40 ml RPMI/10% FBS/1% anti-anti and plated (≈1 mouse/150×25 mm tissue culture dish with 20 mm grid. Media was supplemented with 20 ng/ml GM-CSF and 10 ng/ml IL-4. Media was refreshed on days 3 and 5, and cells were harvested on day 6 and treated accordingly. BMDC were electroporated with siRNA 72 hours prior to injection, and loaded and matured 24 hours prior to ipsilateral footpad injection into B16-treated recipient mice. Recipient mice received 50,000 B16 cells subcutaneously on the flank 72 hours prior DC injection (so that tumors were barely palpable at time of DC administration). DC injection was accompanied by peri-tumoral adjuvantation with 500 ug/mouse imiquimod (Sigma). Mice were boosted in the ipsilateral footpad on day 14 in conjunction with additional imiquimod adjuvantation. Tumors were measured by caliper every other day.

### Example 2—Confirmation and Characterization of CTLA-4 Expression in DC

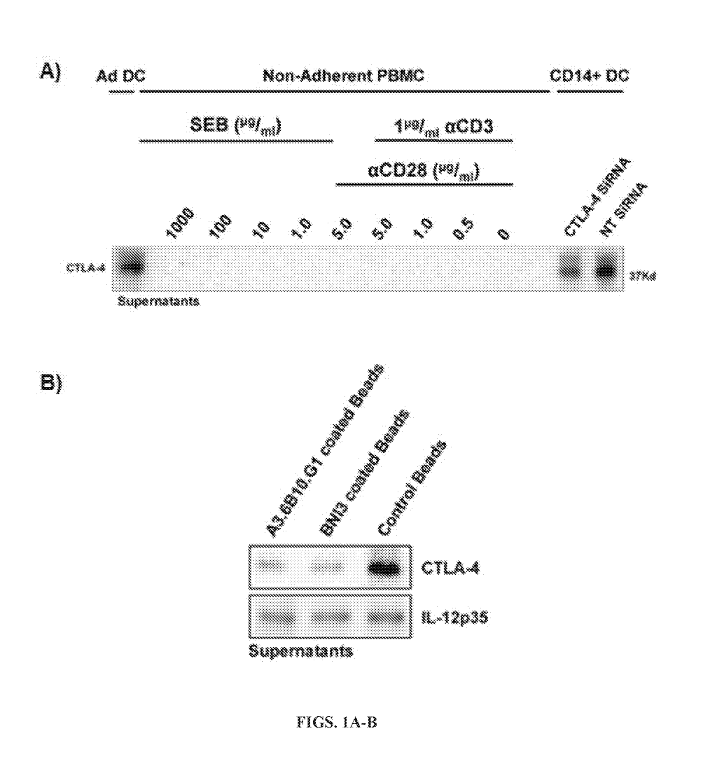

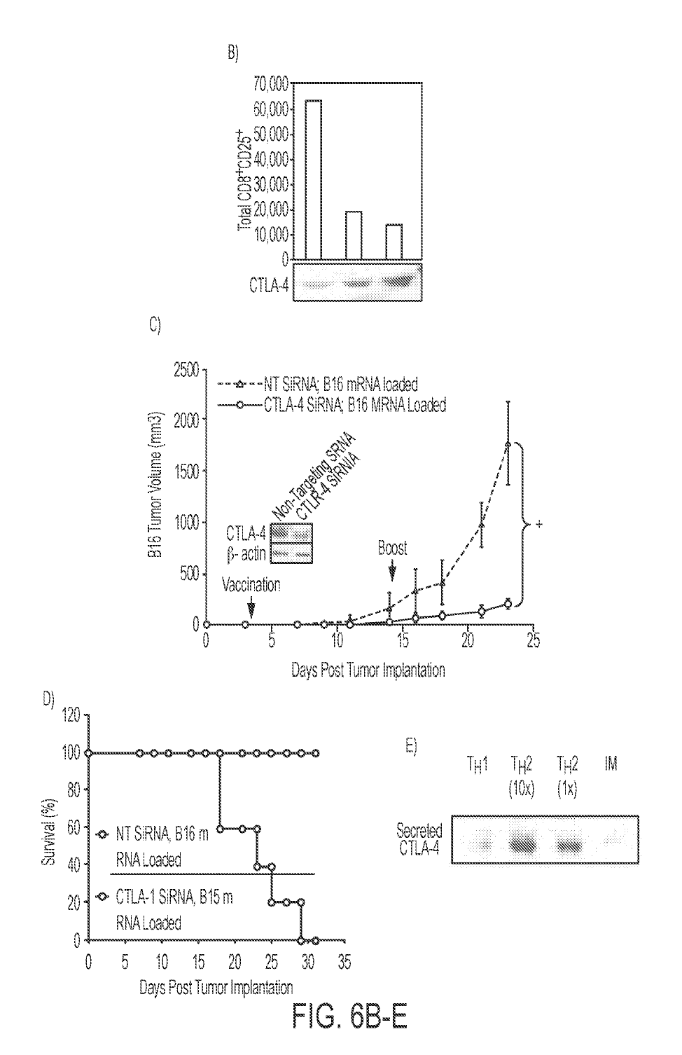

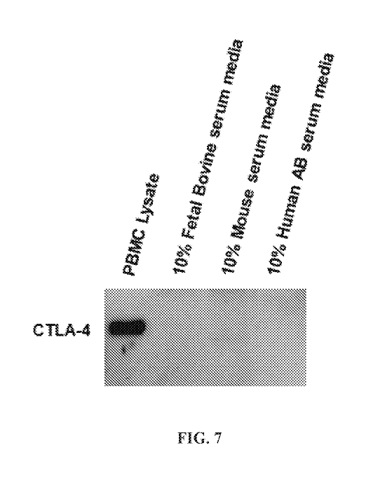

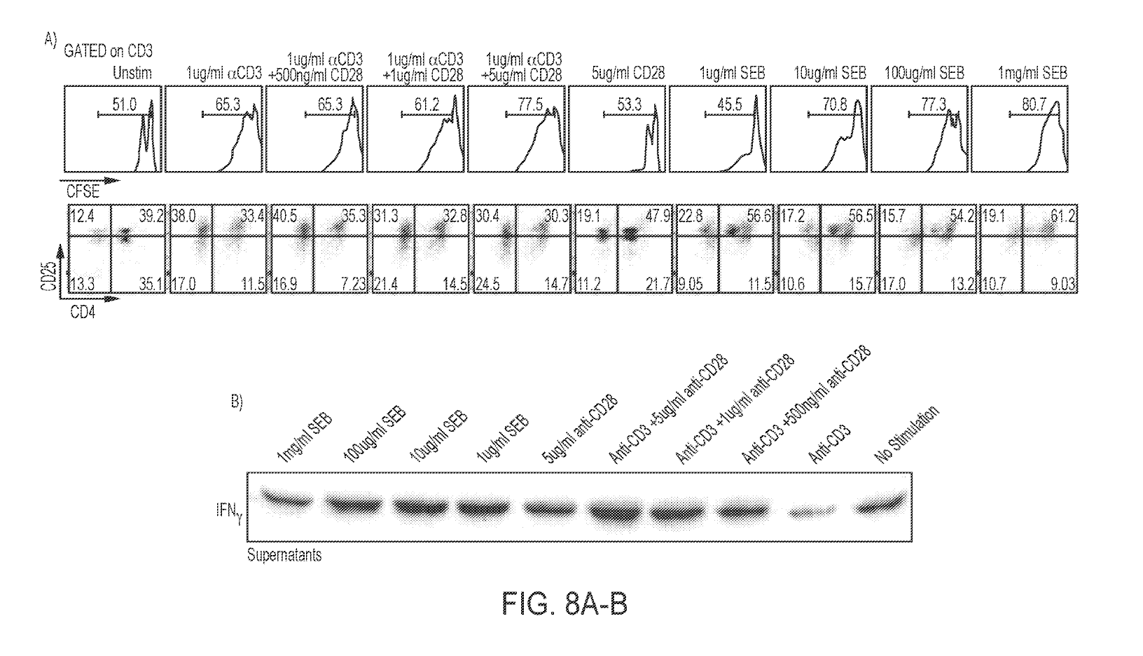

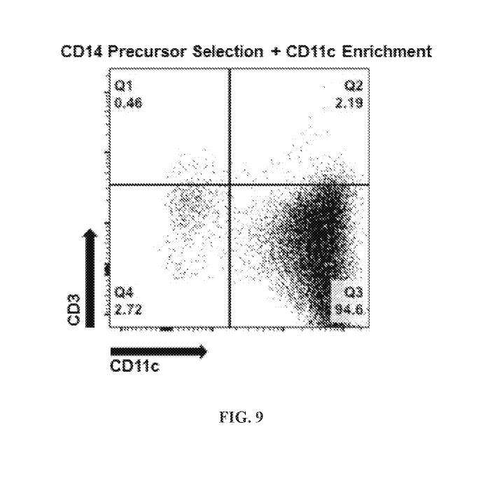

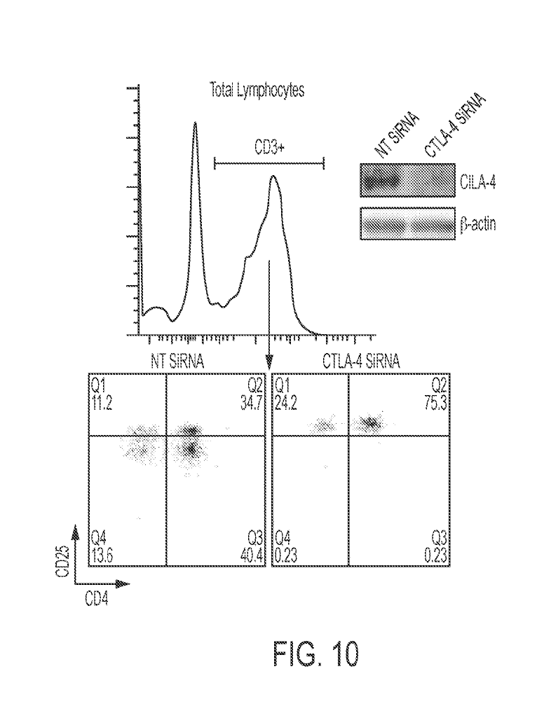

The inventors have previously reported that monocyte-derived DC express the CTLA-4 mRNA transcript yet do not exhibit detectable CTLA-4 on the cell surface (Decker et al., 2009). Several other sporadic reports have also suggested expression of CTLA-4 by DC or CD14⁺ myeloid cells under a variety of different conditions; however, conclusive characterization has been elusive (Han et al., 2014; Wang et al., 2011; Laurent et al., 2010; Pistillo et al., 2003). Given that CTLA-4 surface expression was undetectable, the inventors sought to test the hypotheses that DC expression of CTLA-4 could be intracellular, secretory, or a combination of both possibilities. To determine whether or not DC secrete CTLA-4, the inventors analyzed the culture medium of several different matured DC preparations as well as that of cultured syngeneic non-adherent PBMC (peripheral blood mononuclear cells) by western blot analysis, detecting CTLA-4 only in the culture medium of DC preparations (FIG. 1A). After verifying that CTLA-4 was not an inherent component of the culture media itself (FIG. 7), the inventors further verified the identity of the presumed CTLA-4 western blot band by siRNA knockdown of CTLA-4 (also FIG. 1A) as well as by performing a CTLA-4-specific depletion of the cell culture medium using beads covalently bound to one of two different well-characterized αCTLA-4 clones (BNI3 and A3.6B10.G1). Each bead-bound antibody clone was independently able to substantially abrogate the CTLA-4 band detected by western blot whereas beads bound to an irrelevant isotype control antibody were not (FIG. 1B). None of the bead-bound antibodies diminished the signal of non-specific proteins like IL-12 (also FIG. 1B). Interestingly, the CTLA-4 isoform most prominently found in the culture media migrated just above the 37 kd molecular weight marker, the previously-reported size of the full-length (flCTLA-4) isoform containing both the cytoplasmic and transmembrane domains. In contrast, CTLA-4 secretion was not detected from CD14⁻ PBMC under native or hyperstimulatory conditions (FIG. 1A) despite significantly increased proliferation, activation and IFN-γ release (FIGS. 8A-B) under such conditions. Further, the inventors demonstrated that the DC preparations generated by CD14-selection and subsequent CD11c enrichment were virtually devoid of CD3⁺ cells (FIG. 9), suggesting the source of secreted CTLA-4 was indeed a CD11c⁺CD3⁻ cell type. To confirm that CTLA-4 siRNA was truly targeting CTLA-4, non-adherent PBMC were transfected with the identical CTLA-4 siRNA pool and the predicted functional consequence (i.e. activation) of CTLA-4 knockdown in CD3⁺ T-cells was verified (FIG. 10). Monocyte-derived DC express and secrete CTLA-4.

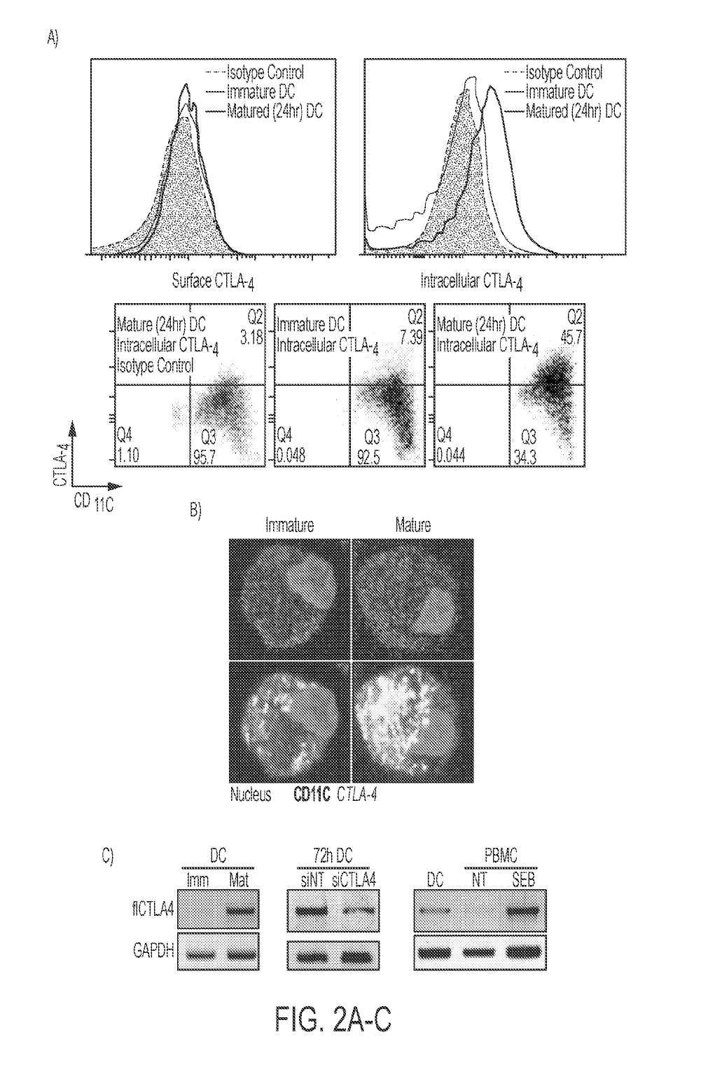

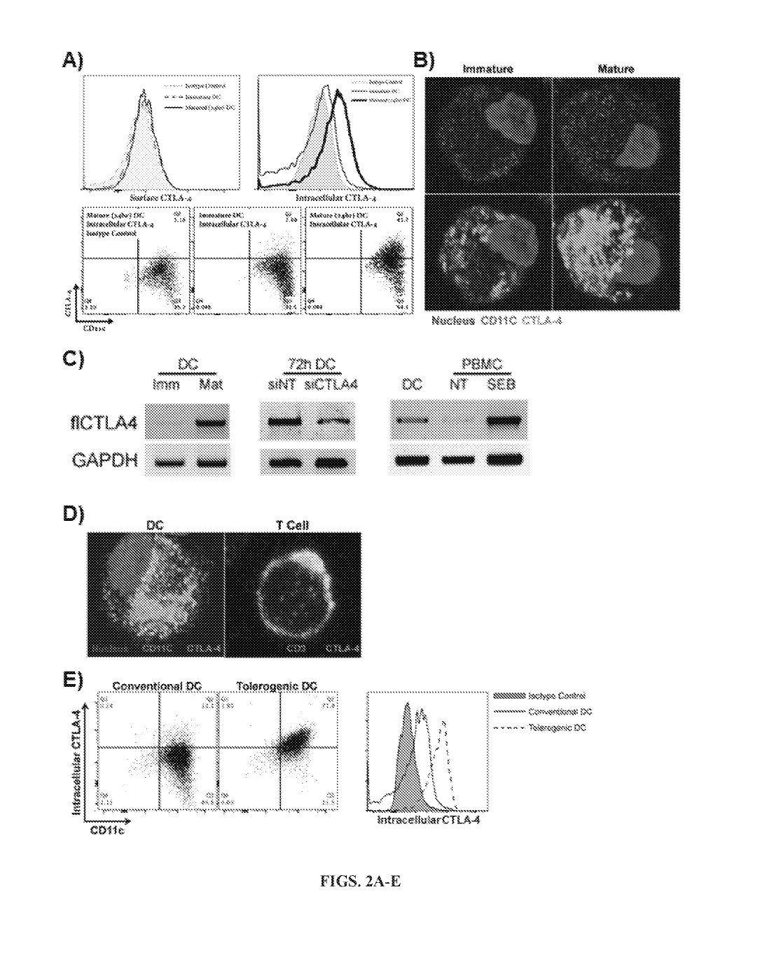

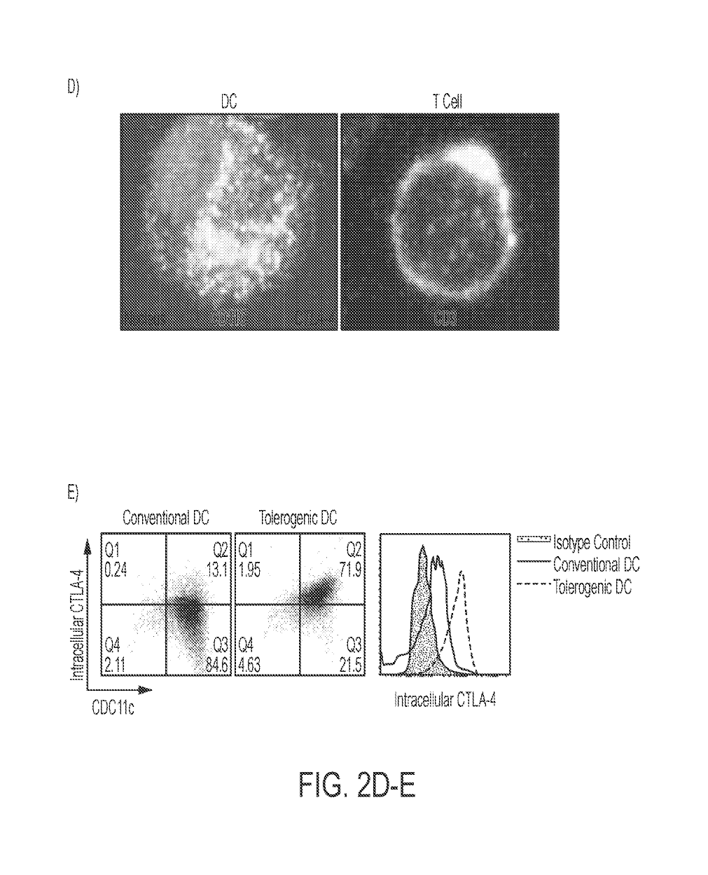

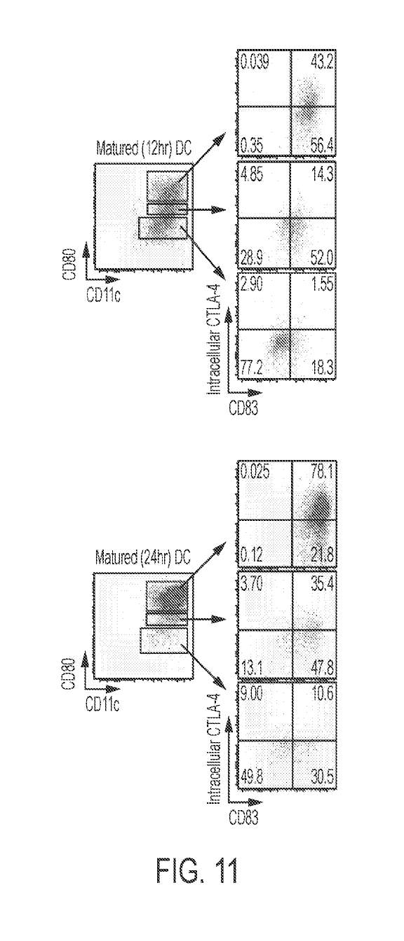

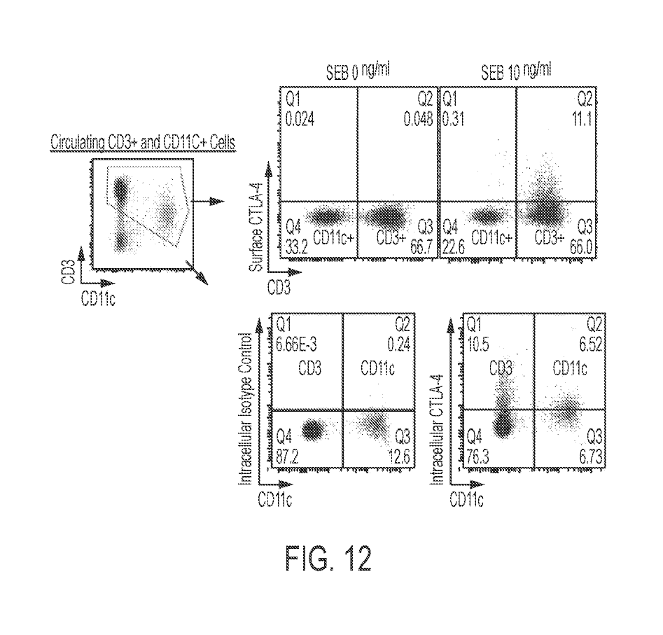

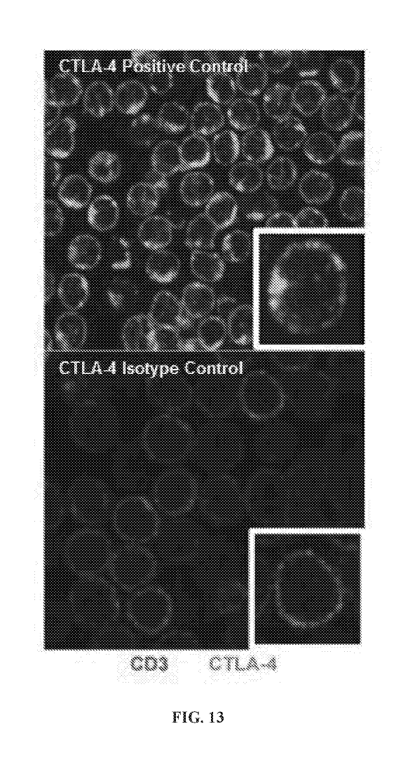

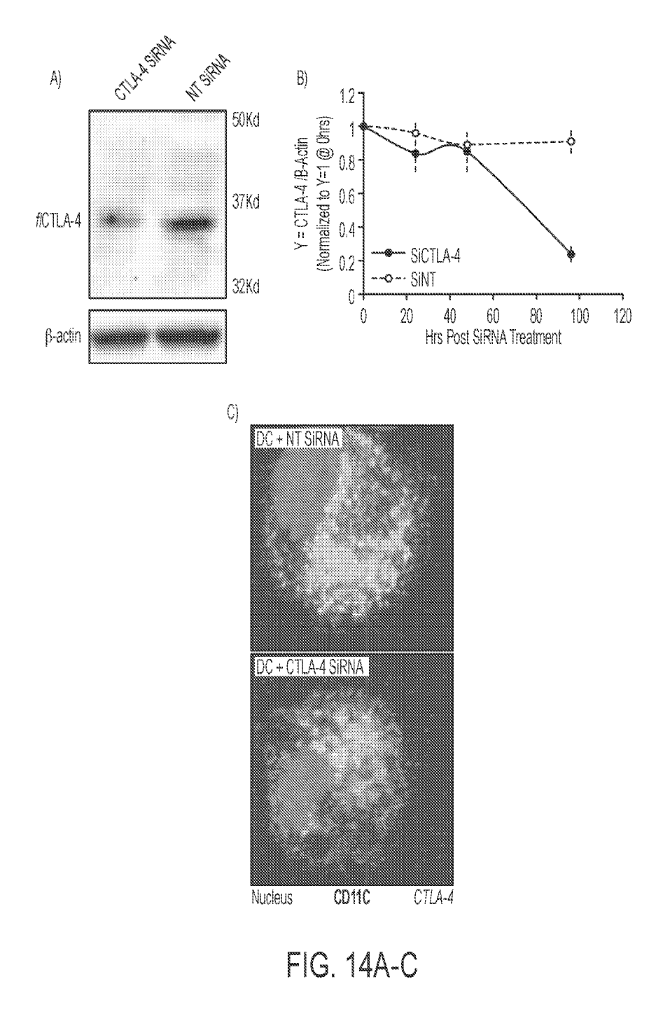

Given the presence of secreted CTLA-4 attributable to DC, the inventors presumed to find CTLA-4 on or within the DC itself. Though they were unable to routinely detect CTLA-4 on the surface of immature or mature DC, the inventors identified CTLA-4 intracellularly by flow cytometry (FIG. 2A) and confocal microscopy (FIG. 2B). DC were observed to express significantly more CTLA-4 as they matured (FIG. 2A, 2B), an observation supported by RT-PCR (FIG. 2C). Indeed, upregulation of CTLA-4 expression was closely correlated with that of other maturation markers like CD80 and CD83 (FIG. 11). In comparison to activated T-cells, the pattern of CTLA-4 localization appeared distinctly different, dispersed throughout the inside of the cell rather than concentrated near the plasma membrane (FIG. 2D). Moreover, staining of tolerizing or “tolerogenic” DC generated in the presence of M-CSF and TGF-β (Li et al., 2007) indicated a CD11c⁺ cell population with log-fold higher CTLA-4 expression levels than conventional DC generated with GM-CSF (FIG. 2E). Further, circulating CD11c⁺ cells harvested from healthy donors displayed comparable intracellular CTLA-4 levels with donor-matched circulating CD3⁺ cells, supporting in vivo physiologic relevance (FIG. 12); isotype control antibody indicated excellent staining specificity (FIG. 13). siRNA knockdown of CTLA-4 led to a significant diminution of signal over a period of five days as indicated by both Western blot (FIG. 14A) and confocal microscopy (FIG. 14C). Interestingly, CTLA-4 in DC appeared to be quite stable with nearly all diminution of signal observed between 48 and 96 hours post-siRNA administration (FIG. 14B). This contrasted significantly with the stability of T-cell CTLA-4, the knockdown of which was nearly complete within 24 hours of siRNA administration.

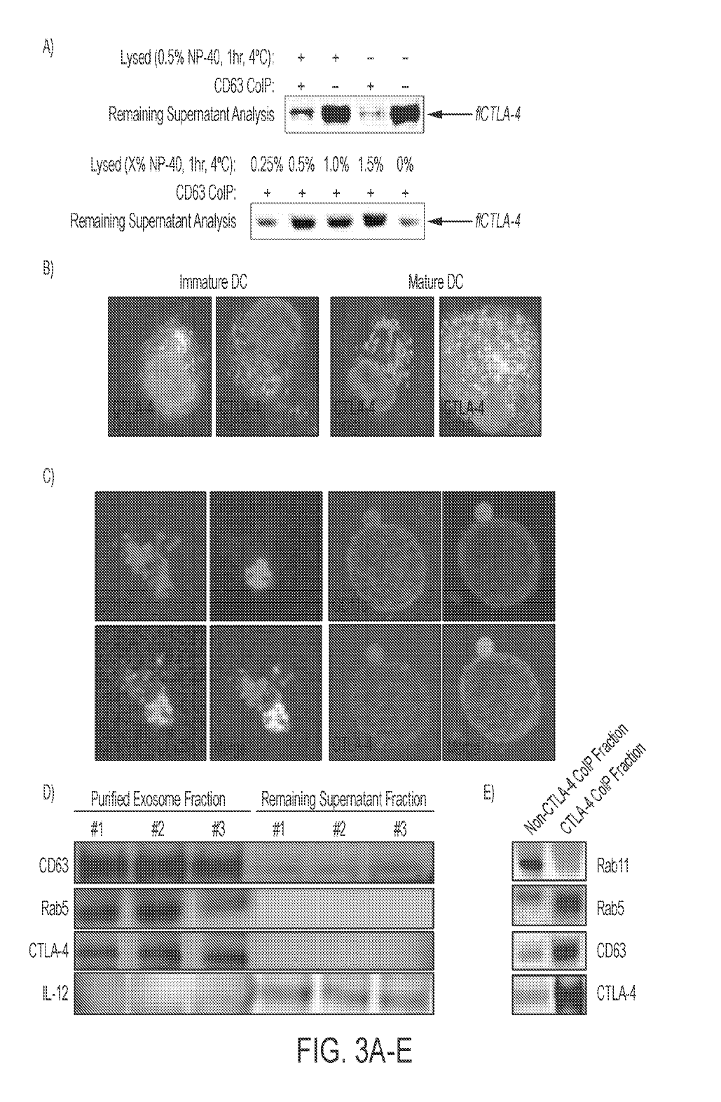

Despite detection of a CTLA-4 isoform the appropriate size of soluble CTLA-4 (i.e. expressed without the transmembrane domain, data not shown), this was not the predominant isoform of CTLA-4 detected in DC culture media. Rather, the vast majority of detected CLTA-4 corresponded in predicted size to the full length (flCTLA-4) isoform. DC have been reported to communicate with other cells through the directed secretion of microvesicles containing numerous ligands, receptors, and other molecules (Sobo-Vujanovic et al., 2014). Since microvesicles possess lipid membranes, it would be feasible for flCTLA-4 to be secreted by means of DC microvesicle release. If this were the case, then depletion of microvesicles by coIP should also deplete CTLA-4 from the culture supernatants. LAMP-3 (lysosomal associated membrane protein 3) or CD63 is an endosomal marker and has also been implicated as one of the most abundant proteins found on the surface of circulating microvesicles/exosomes (Wiley and Gummuluru, 2006). CD63 coIP of DC cell culture supernatant almost completely abrogated the CTLA-4 signal previously seen by western blot (FIG. 3A), indicating that removal of CD63⁺ microvesicles from the DC culture media was sufficient to also remove observed flCTLA-4. Partial lysis of the exosomal fraction prior to CD63 coIP restored some flCTLA-4 signal, presumably because lysis of microvesicular lipid membranes freed some CTLA-4 from CD63-containing vesicles. Lysis in the absence of CD63 coIP did not affect the amount of CTLA-4 detected in the media, eliminating the possibilities that the CD63 antibody non-specifically removed CTLA-4 or that the lysis procedure interfered with western blot detection. To further confirm that the flCTLA-4 observed in the extracellular milieu was localized within CD63-containing microvesicles, supernatants were lysed with increasing concentrations of NP-40 lysis buffer for 1 hr, depleted of remaining microvesicles by CD63 coIP, and analyzed for remaining CTLA-4 content by western blot. As shown, increasing concentrations of lysis buffer lead to a more intense flCTLA-4 signal via western blot, on par with that of supernatants not depleted of exosomes by CD63 coIP (FIG. 3A).

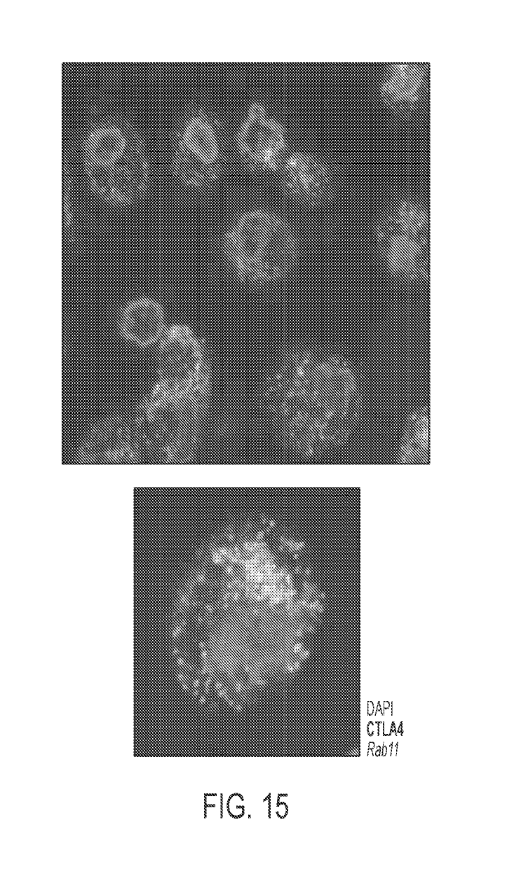

The presence of extracellular CTLA-4 in secretory vesicles should correspond with the presence of intracellular CTLA-4 colocalized with components of the secretory machinery. Confocal microscopy indicated good colocalization of intracellular CTLA-4 within the Golgi apparatus of immature DC (FIG. 3B). Upon maturation, CTLA-4 colocalization migrated from the Golgi to Rab5, a small GTPase known to be a master regulator of endosome biogenesis and a marker that identifies secretory endosomes (FIG. 3B/3C) (Azouz et al, 2014). Rab11, a marker of recycling endosomes (Ullrich et al., 1996), was not observed to colocalize with CTLA-4 to any significant degree (FIG. 15). Colocalization with the Golgi in immature DC or with Rab5 in mature DC was mutually exclusive. Confocal data indicated that CTLA-4 colocalization with Rab5 could occur in a highly polarized fashion (FIG. 3C, left panel) within the cytoplasm as well as within secretory export vesicles in the process of budding (FIG. 3C, right panel). Exosomal microvesicles 30-120 nm in size were purified from DC supernatants derived from three independent biological samples using the Total Exosome Isolation Kit, and efficiency of the exosome isolation protocol was analyzed by comparing the remaining supernatant to the exosomal fraction for the presence of CD63. While the soluble supernatant fraction continued to contain secreted proteins such as IL-12, the exosomal fraction Rab5 and CTLA-4 were localized exclusively within the exosomal fraction (FIG. 3D). CTLA-4 coIP of the purified exosomes and subsequent analysis of the two fractions indicated that CTLA-4 did indeed colocalize extracellularly with Rab5 but not Rab11, similar to what was observed intracellularly by confocal microscopy (FIG. 3E). Taken together, the data indicate that flCTLA-4 is packaged for secretion in immature DC, becomes associated with the active secretory machinery upon maturation, and is ultimately secreted into the extracellular environment within intact microvesicles, exosomal in nature.

### Example 3—Characterization of DC CTLA-4 Function

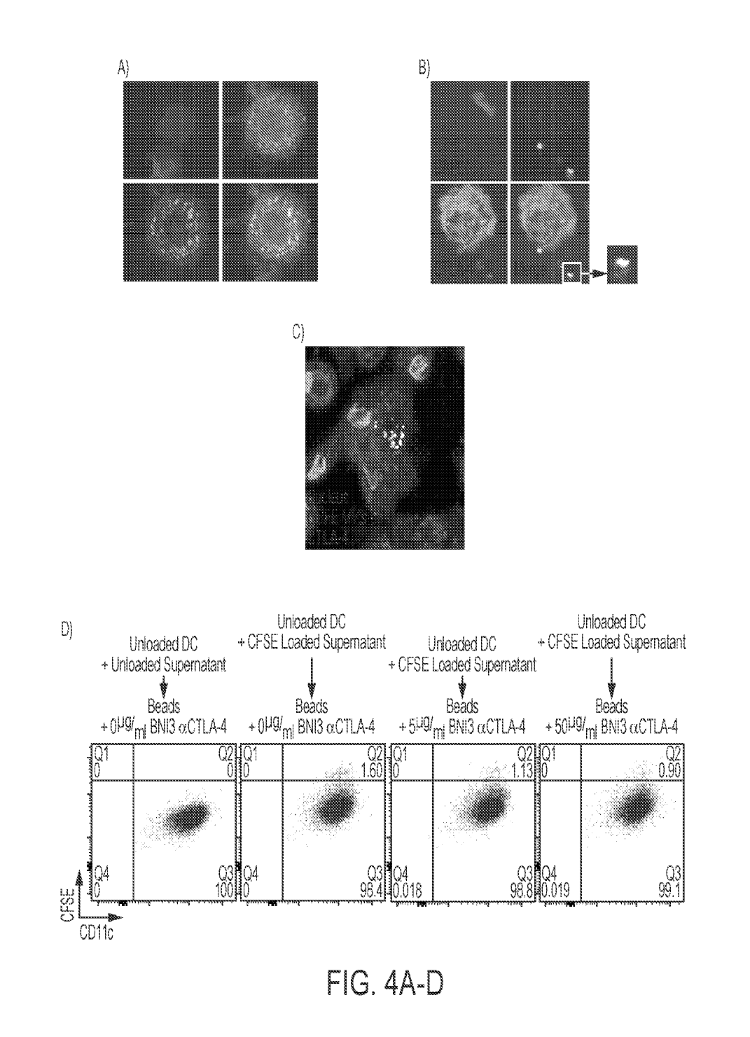

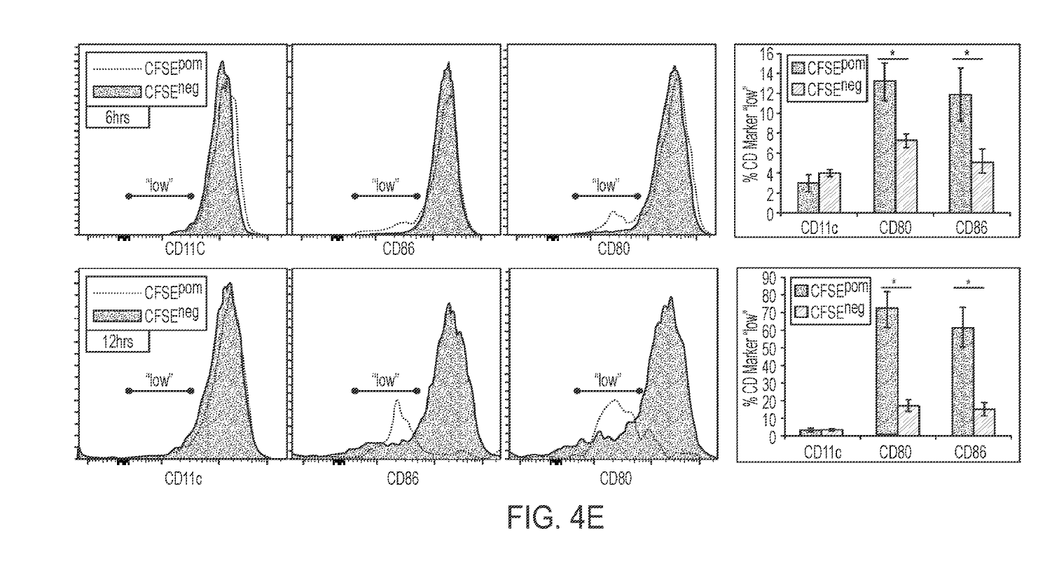

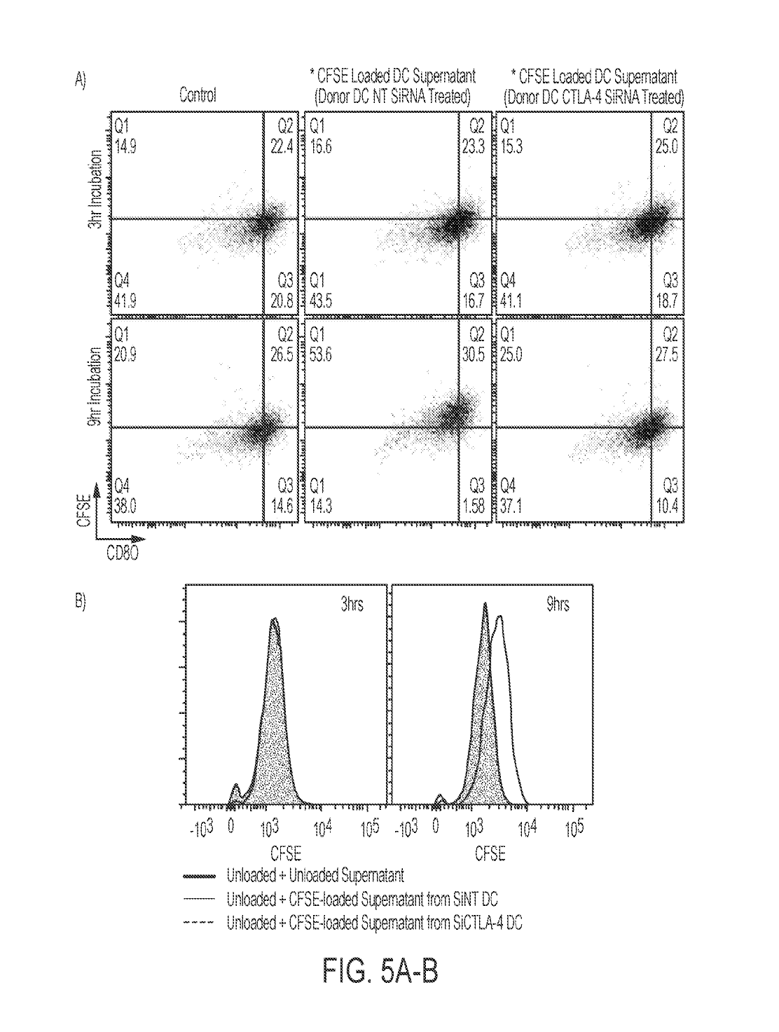

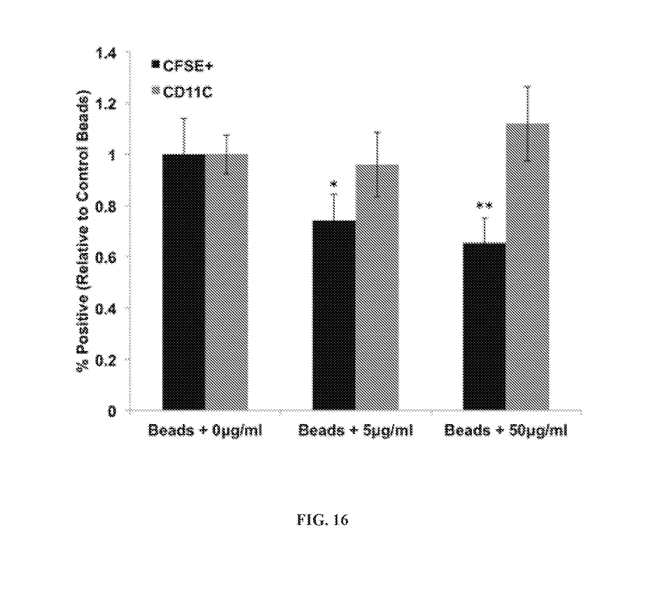

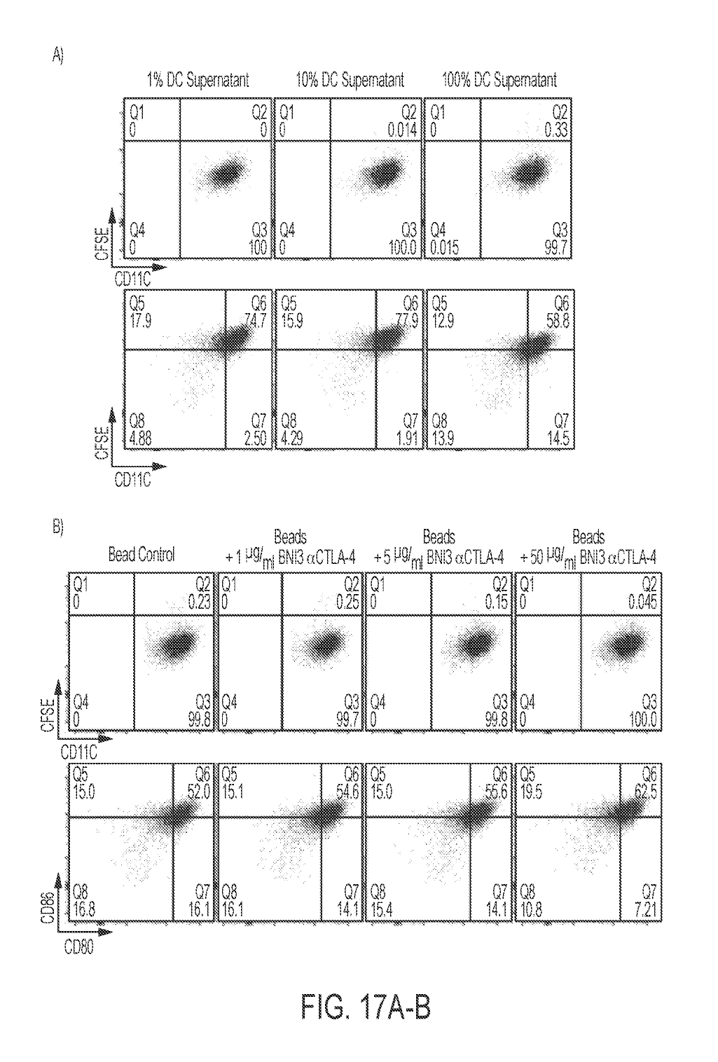

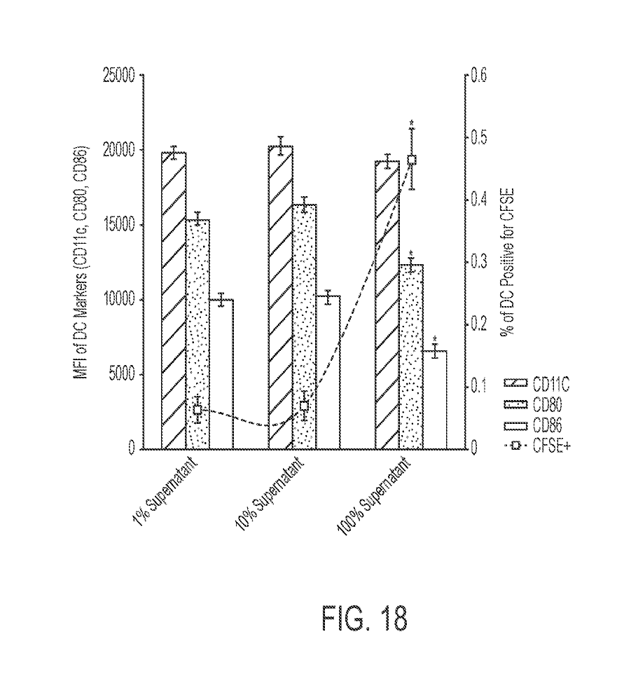

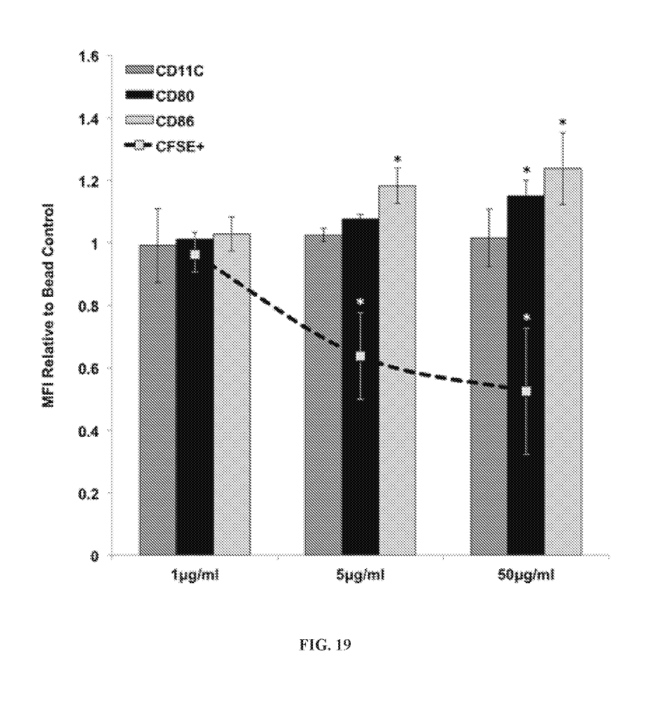

To ascertain functional significance of DC-secreted microvesicles, the inventors first sought to determine if such vesicles could be internalized. To ascertain this, DC were labeled with CFSE. FIG. 4A demonstrates that CFSE uptake by DC was relatively uniform throughout the cell and also colocalized significantly with CTLA-4⁺ endosomes. CFSE-labeled DC were then cultured for 48 hours during which time CFSE⁺ microvesicles were secreted into the culture supernatant. CFSE⁺ supernatants were then harvested and added to unlabeled DC onto which CFSE⁺ microvesicles could subsequently be shown to bind (FIG. 4B) and ultimately be internalized (FIG. 4C), a process that could be followed over time by both confocal microcopy (FIGS. 4B-C) and flow cytometry (i.e. FIGS. 4D and 16). Pre-clearance of supernatants with beads conjugated to anti-CTLA-4 clone BNI3 could reduce uptake of CFSE-labeled microvesicles by unlabeled DC in a fashion dependent upon the concentration of bead-conjugated BNI3 antibody (FIGS. 4D and 16). Because microvesicle uptake could easily be quantitated by flow cytometry, the consequences of such uptake on B7 surface expression could be monitored as well. As indicated by FIG. 4E, DC that became CFSE positive exhibited log-fold lower levels of CD80 and CD86 surface expression with little to no change observed in the expression of other surface markers such as CD11c. The process of B7 diminution was time-dependent. Though no uptake was observed after 3 hours of incubation, after 6 hours of incubation in CFSE-labeled microvesicles, 12-13% of CFSE⁺ DC were B7 “low” (in comparison to 5-6% of CFSE⁻ DC); however, after 12 hours of incubation, 65-75% of CFSE DC were B7 “low” (in comparison to 15% of CFSE⁻ DC). Staining of B7 for 20 minutes at 4° C. in the presence of 0.1% sodium azide (i.e. conditions under which B7 receptor downregulation could not occur) in 100% DC supernatant indicated the presence of a titratable factor in the supernatant that reduced the amount of antibody binding to B7 without reducing the amount of antibody binding to CD11c (FIGS. 17A and 18). As with previous experiments, this factor could be removed by preclearance with beads conjugated to anti-CTLA-4 BNI3 (FIGS. 17B and 19). To further characterize the dependency of microvesicle uptake upon CTLA-4, DC were first treated with either non-targeting (NT) siRNA or CTLA-4-specific siRNA prior to CFSE-labeling and DC maturation. As shown in FIGS. 5A and B, after 9 hours of incubation in CFSE-labeled supernatants derived from CTLA-4 siRNA-treated DC, recipient DC remained predominantly CFSE⁻, whereas recipient DC treated with CFSE-labeled supernatants derived from NT siRNA-treated DC became CFSE⁺, suggesting that microvesicle uptake was dependent upon CTLA-4/B7 interaction.

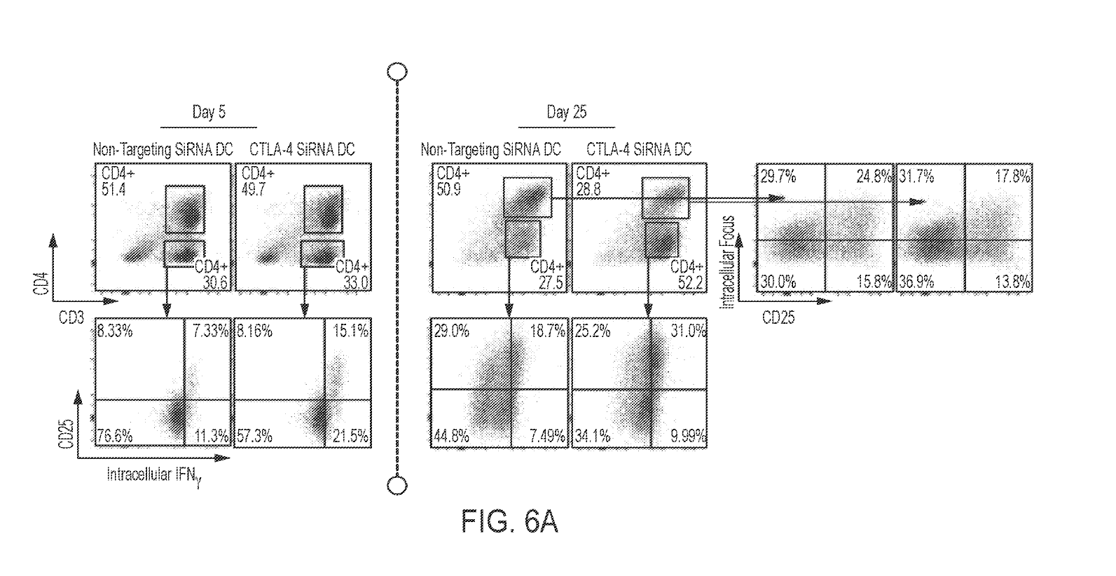

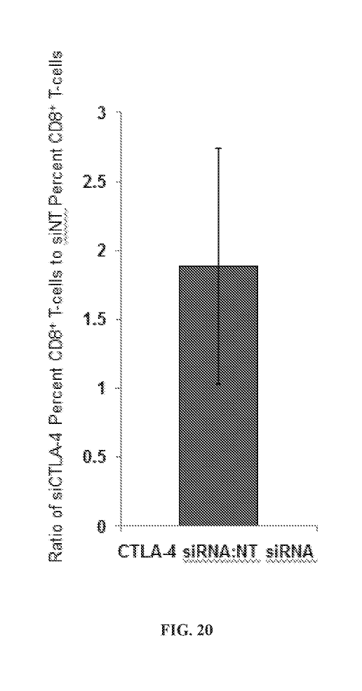

Since there are few reports on the existence and/or function of DC CTLA-4, we next assayed whether or not DC CTLA-4 was functional relative to the canonical understanding of CTLA-4 biology. To test whether DC-secreted CTLA-4 negatively regulates T-cells activation, PBMC were cocultured with DC treated either with NT siRNA or CTLA-4 siRNA (for 72 hours prior coculture initiation). Though the CD8:CD4 ratios were similar at early time points (e.g. day 5), the CD8⁺CD25⁺IFN-γ⁺ fraction was much greater when PBMC were cultured with DC lacking CTLA-4 (FIG. 6A, left panel). Following the early trend, by day 25 T-cells cultured with CTLA-4-deficient DC consistently exhibited a significant increase in the CD8:CD4 ratio with a greater percentage of CD8⁺CD25⁺IFN-γ⁺ cells (FIGS. 6A and 20). Additionally, the percentage of CD4⁺CD25⁺Foxp3⁺ tregs was significantly less when DC lacked CTLA-4 (FIG. 6A, right panel). Similarly, incubation of total splenocytes in siRNA-treated DC culture supernatants possessing differing amounts of CTLA-4⁺ microvesicles demonstrated that subsequent proliferation of CD8⁺CD25⁺ cells was dependent upon low supernatant CTLA-4 content as well as proportional to the concentration of CTLA-4 in the supernatant (FIG. 6B). To test physiological relevance of DC CTLA-4 in vivo, a B16 melanoma DC-vaccine study was conducted in which the only variable altered was the addition of either NT or CTLA-4 siRNA 48 hrs prior to electroporation of DC with B16 mRNA. Following electroporation, DC were matured for 24 hours, and injected into recipient mice with pre-established, palpable B16 tumors. Mice that received the CTLA-4 siRNA DC vaccine exhibited significantly delayed tumor growth (FIG. 6C) decreased metastasis, and increased survival (FIG. 6D). Given the association of DC CTLA-4 knockdown with enhanced production of CD8⁺IFN-γ⁺ cells and augmented antitumor immunity, we sought to determine if TH polarization might play a role in DC CTLA-4 release. Maturing DC were incubated in either high dose IL-12 to induced TH1 polarization or SEB (Mandron et al, 2006) to induce TH2 polarization, and CTLA-4 release was quantitated by western blot analysis of DC culture supernatants. As shown in FIG. 6E, TH1 polarization of DC resulted in a near-complete abrogation of CTLA-4 secretion whereas TH2 polarization resulted in increased CTLA-4 secretion in a dose-dependent fashion. In aggregate, the data suggest that DC CTLA-4 serves a clear functional purpose in the regulation of CD8 CTL activity with concomitant physiologic consequence in tumor immunity.

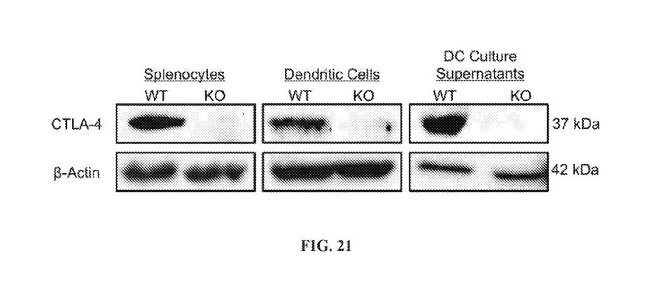

An analysis of cells derived from CTLA-4^{-/-}CD28^{-/-} double knockout mice confirms expression of CTLA-4 in murine DC. Splenocytes, BMDC, and BMDC culture supernatants were generated from wild type and CTLA-4^{-/-}CD28^{-/-} double knockout mice and then analyzed for CTLA-4 content by Western blot analysis (FIG. 21).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,469,797
U.S. Pat. No. 4,472,509
U.S. Pat. No. 4,606,855
U.S. Pat. No. 4,703,003
U.S. Pat. No. 4,742,159
U.S. Pat. No. 4,767,720
U.S. Pat. No. 4,816,567
U.S. Pat. No. 4,867,973
U.S. Pat. No. 4,938,948
U.S. Pat. No. 4,946,778
U.S. Pat. No. 5,021,236
U.S. Pat. No. 5,091,513
U.S. Pat. No. 5,164,296
U.S. Pat. No. 5,196,066
U.S. Pat. No. 5,223,409
U.S. Pat. No. 5,403,484
U.S. Pat. No. 5,420,253
U.S. Pat. No. 5,565,332
U.S. Pat. No. 5,571,698
U.S. Pat. No. 5,627,052
U.S. Pat. No. 5,656,434
U.S. Pat. No. 5,770,376
U.S. Pat. No. 5,789,208
U.S. Pat. No. 5,821,337
U.S. Pat. No. 5,844,091
U.S. Pat. No. 5,858,657
U.S. Pat. No. 5,861,155
U.S. Pat. No. 5,871,907
U.S. Pat. No. 5,969,108
U.S. Pat. No. 6,054,297
U.S. Pat. No. 6,165,464
U.S. Pat. No. 6,365,157
U.S. Pat. No. 6,406,867
U.S. Pat. No. 6,506,559
U.S. Pat. No. 6,573,099
U.S. Pat. No. 6,709,659
U.S. Pat. No. 6,709,873
U.S. Pat. No. 6,753,407
U.S. Pat. No. 6,814,965
U.S. Pat. No. 6,849,259
U.S. Pat. No. 6,861,572
U.S. Pat. No. 6,875,434
U.S. Pat. No. 6,881,557
U.S. Pat. No. 6,891,024
U.S. Pat. No. 6,946,546
U.S. Pat. No. 8,728,806
U.S. Patent App. Pub. 2002/0168707
U.S. Patent App. Pub. 2002/0172677
U.S. Patent App. Pub. 2003/0051263
U.S. Patent App. Pub. 2003/0055020
U.S. Patent App. Pub. 2003/0159161
U.S. Patent App. Pub. 2004/0064842
U.S. Patent App. Pub. 2004/0126828
U.S. Patent App. Pub. 2004/0265839
U.S. Patent App. Pub. 2005/0214860
Azouz et al. J. Immunol. 192:4043-4053, 2014.
Barbas et al., 1994.
Daroszewski et al. Eur. J. Endocrinol. 161:787-793, 2009.
Decker et al. Blood. 113:42113-4223, 2009.
Dejean et al. Nat. Immunol. 10:504-513, 2009.
Esposito et al. J. Immunol. 193:889-900, 2014.
Gardner et al. Am. J. Transplant. 14:1985-1991, 2014.
Gram et al., 1992.
Grohmann et al. Nat. Immunol. 3:1097-1101, 2002.
Han et al. Hepatology. 59:567-579, 2014.
Hodi et al. N. Engl. J. Med. 363:711-723, 2010.
Hu et al., 1996
Kowalczyk et al. Eur. J. Immunol. 44:1143-1155, 2014.
Krummel M F, and Allison J P. J. Exp. Med. 182:459-465, 1995.
Kuehn et al., Science. 345:1623-1627, 2014.
Kuiper et al. J. Immunol. 155:1776-1783, 1995.
Laurent et al. Hum. Immunol. 71:934-941, 2010.
Li et al. Blood. 110:2872-2879, 2007.
Linsley et al. J. Exp. Med. 176:1595-1604, 1992.
Liu et al., 2003.
Mandron et al. J Allergy Clin Immunol 117:1141-1147, 2006.
Marks (1992).
Masteller et al. J. Immunol. 164:5319-5327, 2000.
Oaks M K and Hallett K M. J. Immunol. 164:5015-5018, 2000.
Pistillo et al. Blood. 101:202-209, 2003.
Purohit et al. J. Autoimmune Dis. 2:8, 2005.
Qureshi et al. Science. 332:600-603, 2011.
Read et al. J. Exp. Med. 192:295-302, 2000.
Schier et al., 1996.
Schubert et al. Nat. Med. 20:1410-1416, 2014.
Sobo-Vujanovic et al. Cell Immunol. 289:119-127, 2014.
Sojanovic et al. J. Immunol. 192:4184-4191, 2014.
Stemmer (1994)
Tivol et al. Immunity 3:541-547, 1995.
Ullrich et al. J. Cell Biol. 135:913-924, 1996.
Wang et al. BMC Immunol. 12:21, 2011.
Waterhouse et al. Science. 270:985-988, 1995.
Wiley R D and Gummuluru S. Proc. Natl. Acad. Sci. U.S.A. 103:738-743, 2006.
Wing et al. Science. 322:271-275, 2008.
Wing et al. Trends Immunol. 32:428-433, 2011.

What is claimed is:

1. An immunogenic composition comprising (i) at least a first antigen-primed dendritic cell or antigen and (ii) a CTLA-4 antagonist.

2. The composition of claim 1, wherein the CTLA-4 antagonist is an inhibitory nucleic acid specific to CTLA-4.

3. The composition of claim 2, wherein the inhibitory nucleic acid is a RNA.

4. The composition of claim 3, wherein the RNA is a small interfering RNA (siRNA) or a short hairpin RNA (shRNA).

5. The composition of claim 1, wherein the CTLA-4 antagonist is a CTLA-4-binding antibody.

6. The composition of claim 5, wherein the antibody is a monoclonal antibody.

7. The composition of claim 5, wherein the antibody is recombinant.

8. The composition of any one of claims 5-7, wherein the antibody is an IgG, IgM, IgA or an antigen binding fragment thereof.

9. The composition of any one of claims 5-8, wherein the antibody is a Fab′, a F(ab′)2, a F(ab′)3, a monovalent scFv, a bivalent scFv, or a single domain antibody.

10. The composition of any one of claims 5-9, wherein the antibody is a human antibody, humanized antibody or de-immunized antibody.

11. The composition of claim 1, comprising an antigen-primed dendritic cell.

12. The composition of claim 1, comprising a first antigen, wherein the antigen is a tumor cell antigen or an infectious disease antigen.

13. A method of providing an immune response in a subject comprising administering an immunogenic composition to the subject in conjunction with a CTLA-4 antagonist.

14. The method of claim 13, wherein the CTLA-4 antagonist is an inhibitor nucleic acid specific to CTLA-4.

15. The method of claim 14, wherein the inhibitory nucleic acid is a RNA.

16. The method of claim 15, wherein the RNA is a small interfering RNA (siRNA) or a short hairpin RNA (shRNA).

17. The method of claim 13, wherein the CTLA-4 antagonist is a CTLA-4-binding antibody.

18. The method of claim 17, wherein the antibody is a monoclonal antibody.

19. The method of claim 17, wherein the antibody is recombinant.

20. The method of any one of claims 17-19, wherein the antibody is an IgG, IgM, IgA or an antigen binding fragment thereof.

21. The method of any one of claims 17-20, wherein the antibody is a Fab′, a F(ab′)2, a F(ab′)3, a monovalent scFv, a bivalent scFv, or a single domain antibody.

22. The method of any one of claims 17-20, wherein the antibody is a human antibody, humanized antibody or de-immunized antibody.

23. The method of claim 13, wherein the immunogenic composition comprises an antigen-primed dendritic cell population.

24. The method of claim 13, wherein the immunogenic composition comprises a polypeptide antigen.

25. The method of claim 13, wherein the immunogenic composition comprises a nucleic acid encoding an antigen.

26. The method of claim 25, wherein the nucleic acid is a DNA expression vector.

27. The method of claim 25, wherein immunogenic composition is administered before or essentially simultaneously with the CTLA-4 antagonist.

28. The method of claim 25, wherein immunogenic composition is administered after the CTLA-4 antagonist.

29. The method of any one of claims 27-28, wherein immunogenic composition is administered within about 1 week, 1 day, 8 hours, 4 hours, 2 hours or 1 hour of the CTLA-4 antagonist.

30. The method of claim 13, wherein the immunogenic composition comprises a tumor cell antigen or an infectious disease antigen.

31. The method of claim 13, wherein the immunogenic composition comprises at least a first adjuvant.

32. The method of claim 13, wherein the subject has or is at risk for a disease.

33. The method of claim 32, wherein the disease is an infectious disease or a cancer.

34. A dendritic cell population, wherein said population has been genetically modified to reduce the expression of CTLA-4.

35. The cell population of claim 34, wherein the genetic modification comprises introduction of an exogenous inhibitory nucleic acid specific to CTLA-4.

36. The cell population of claim 35, wherein the inhibitory nucleic acid is a RNA.

37. The cell population of claim 36, wherein the RNA is a small interfering RNA (siRNA) or a short hairpin RNA (shRNA).

38. The cell population of claim 34, wherein the genetic modification comprises a genomic deletion or insertion in the cell population that reduces CTLA-4.

39. The cell population of claim 34, wherein the genetic modification comprises a genomic edit using a CRISPR/Cas nuclease system.

40. The cell population of claim 34, wherein the cell population comprises a hemizygous deletion within the CTLA-4 gene.

41. The cell population of claim 34, wherein the dendritic cells have been primed with at least a first antigen.

42. The cell population of claim 34, wherein the antigen is tumor cell antigen or an infectious disease antigen.

43. A method of providing an immune response in a subject comprising administering an effective amount of a cell population according to anyone of claims 34-42 to the subject.

44. The method of claim 43, wherein the dendritic cells have been primed with at least a first antigen.

45. The method of claim 43, wherein the subject has a cancer and the dendritic cells have been primed with at least a first cancer cell antigen.

46. The method of claim 43, wherein the subject has an infectious disease and the dendritic cells have been primed with at least a first infectious disease antigen.

47. A method culturing antigen specific T-cells comprising culturing a population of T-cells or T-cell precursors in the presence of a population of antigen presenting cells that have been primed with at least a first antigen, wherein:
(i) said culturing is in the presence of a CTLA-4 antagonist; or
(ii) said population of antigen presenting cells comprise a dendritic cell population cell population that has been has been genetically modified to reduce the expression of CTLA-4.

48. The method of claim 47, further defined as a method for ex vivo expansion of antigen specific T-cells.

49. The method of claim 47, wherein the dendritic cell population comprises primary dendritic cells.

50. The method of claim 47, wherein said culturing is in the presence of a CTLA-4 antagonist.

51. The method of claim 50, wherein the CTLA-4 antagonist is an inhibitor nucleic acid specific to CTLA-4.

52. The method of claim 51, wherein the inhibitory nucleic acid is a RNA.

53. The method of claim 52, wherein the RNA is a small interfering RNA (siRNA) or a short hairpin RNA (shRNA).

54. The method of claim 50, wherein the CTLA-4 antagonist is a CTLA-4-binding antibody.

55. The method of claim 47, wherein said dendritic cell population has been has been genetically modified to reduce the expression of CTLA-4.

56. The method of claim 55, wherein the genetic modification comprises introduction of an exogenous inhibitory nucleic acid specific to CTLA-4.

57. The method of claim 56, wherein the inhibitory nucleic acid is a RNA.

58. The method of claim 56, wherein the RNA is a small interfering RNA (siRNA) or a short hairpin RNA (shRNA).

59. The method of claim 55, wherein the genetic modification comprises a genomic deletion or insertion in the cell population that reduces CTLA-4.

60. The method of claim 55, wherein the cell population comprises a hemizygous deletion within the CTLA-4 gene.


SEQUENCE LISTING (2 sequences)

SEQ ID NO: 1 - DNA, 25 bp, Artificial sequence
  Synthetic primer
atggcttgccttggatttcagcggc

SEQ ID NO: 2 - DNA, 27 bp, Artificial sequence
  Synthetic primer
tcaattgatgggaataaaataaggctg